(19) United States
(12) Patent Application Publication
(10) Pub. No.: US 2014/0154751 A1
(43) Pub. Date: Jun. 5, 2014

(54) ENHANCED FERMENTATION OF CELLODEXTRINS AND BETA-D-GLUCOSE

(71) Applicants: James H. Doudna Cate, Berkeley, CA (US); William T. Beeson, IV, Oakland, CA (US); Jonathan M. Galazka, Berkeley, CA (US); Huimin Zhao, Champaign, IL (US); Sijin Li, Urbana, IL (US); Yong-Su Jin, Champaign, IL (US); Suk-Jin Ha, Savoy, IL (US)

(72) Inventors: James H. Doudna Cate, Berkeley, CA (US); William T. Beeson, IV, Oakland, CA (US); Jonathan M. Galazka, Berkeley, CA (US); Huimin Zhao, Champaign, IL (US); Sijin Li, Urbana, IL (US); Yong-Su Jin, Champaign, IL (US); Suk-Jin Ha, Savoy, IL (US)

(73) Assignees: The Regents of the Univeristy of California, Oakland, CA (US); The Board of Trustee of the University of Illinois, Urbana, IL (US)

(21) Appl. No.: 13/980,866

(22) PCT Filed: Jan. 20, 2012
(86) PCT No.: PCT/US12/22079

§ 371 Date: Feb. 14, 2014

Related U.S. Application Data
(60) Provisional application No. 61/435,216, filed on Jan. 21, 2011.

(51) Int. Cl. C12P 19/12; C12P 7/10; C12P 19/14

(52) U.S. Cl. CPC C12P 19/12; C12P 19/14; C12P 7/10
USPC 435/99; 435/100; 435/134; 435/148; 435/157; 435/160; 435/165; 435/166; 435/254.21

(57) ABSTRACT

The present disclosure provides compositions and methods for the fermentation of cellodextrins and β-D-glucose. Host cells and recombinant polypeptides having glucose mutarotase activity are provided. Additionally, methods for improving cell growth, production of chemicals, and consumption of cellodextrins and β-D-glucose during fermentation of mixtures containing cellodextrins and β-D-glucose are provided.

### CROSS-REFERENCE TO RELATED APPLICATIONS

This application claims the benefit of U.S. Provisional Application No. 61/435,216, filed Jan. 21, 2011, which is hereby incorporated by reference in its entirety.

### FIELD

The present disclosure relates to the fermentation of cellodextrins and β-D-glucose. In particular, the present disclosure relates to compositions for the fermentation of cellodextrins and β-D-glucose, including recombinant polypeptides and host cells including recombinant nucleotides and polypeptides, and methods of use thereof. The disclosure further relates to methods for the fermentation of cellodextrins and β-D-glucose.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 658012001040SEQLIST.txt, date recorded: Jan. 11, 2012, size: 61 KB).

### BACKGROUND

Biofuels are under intensive investigation due to the increasing concerns about energy security, sustainability, and global climate change. Bioconversion of plant-based materials into biofuels is regarded as an attractive alternative to chemical production of fossil fuels.

Cellulose, a major component of plants and one of the most abundant organic compounds on earth, is a polysaccharide composed on long chains of β(1-4) linked D-glucose molecules. Due to its sugar-based composition, cellulose is a rich potential source material for the production of biofuels. For example, sugars may be fermented into biofuels such as ethanol. In order for the sugars within cellulose to be used for the production of biofuels, the cellulose must be broken down into smaller molecules.

Cellulose may be enzymatically hydrolyzed by the action of cellulases. Cellulases include endoglucanases, exoglucanases, and beta-glucosidases. The actions of cellulases cleave the 1-4 β-D-glycosidic linkages in cellulose, and result in the ultimate release of β-D-glucose molecules. During the breakdown of cellulose into individual sugar molecules, glucose polymers of various lengths may be formed as intermediate breakdown products. Glucose polymers of approximately 2-6 molecules in length derived from the hydrolysis of cellulose are referred to as “cellodextrins”.

Saccharomyces cerevisiae, also known as baker's yeast, has been used for bioconversion of hexose sugars into ethanol for thousands of years. It is also the most widely used microorganism for large scale industrial fermentation of D-glucose into ethanol. S. cerevisiae is a very suitable candidate for bioconversion of plant-based biomass into biofuels. It has a well-studied genetic and physiological background, ample genetic tools, and high tolerance to high ethanol concentration. The low fermentation pH of S. cerevisiae can also prevent bacterial contamination during fermentation.

Recently, a new strategy to co-ferment mixed sugars present in cellulosic hydrolyzates using S. cerevisiae was demonstrated (Ha et al., Proc. Natl. Acad. Sci. U.S.A. 108:504-509, 2011; Li et al., Mol. Biosyst. 6 (11):2129-2132, 2010). This approach not only facilitated simultaneous co-fermentation of cellobiose and xylose, but also led to improved ethanol yields and productivities due to synergistic effects of cellobiose and xylose co-fermentation. Compared to glucose fermentation, cellobiose fermentation by engineered S. cerevisiae via intracellular hydrolysis has several advantages. First, cellobiose can be co-consumed with other sugars such as xylose or galactose without glucose repression. Second, co-utilization of cellobiose with other sugars could increase overall productivity. Third, expensive β-glucosidase is not necessary for complete cellulose degradation to glucose.

Despite the above-mentioned advantages, the cellobiose consumption rate is much slower than the glucose consumption rate. In addition, small amounts of glucose and the cellodextrins cellotriose and cellotetraose were accumulated in the medium during cellobiose fermentation. Cellotriose and cellotetraose are produced by the transglycosylation activity of β-glucosidase when concentrations of intracellular cellobiose and glucose are high. These observations suggest that there might be unknown limiting steps for efficient cellobiose fermentation by engineered S. cerevisiae.

Disclosed herein are improved compositions and methods for the fermentation of cellodextrins and β-D-glucose molecules by microorganisms.

### BRIEF SUMMARY

Certain embodiments of the present disclosure meet this need by providing recombinant polypeptides having glucose mutarotase activity, host cells including recombinant DNA encoding polypeptides having glucose mutarotase activity, and methods of their production and use. Certain embodiments of the present disclosure meet this need by providing methods for the fermentation of cellodextrins and β-D-glucose molecules, methods for increasing production of a chemical, and methods for increasing growth rate of a cell.

In one embodiment, the disclosure provides a host cell including recombinant DNA encoding one or more cellodextrin transporters and recombinant DNA encoding one or more polypeptides having glucose mutarotase activity, wherein the host cell consumes more molecules of cellobiose when grown in a cellobiose-containing medium than are consumed by a corresponding host cell lacking said recombinant DNA encoding one or more polypeptides having glucose mutarotase activity.

In another embodiment, the disclosure provides a host cell including recombinant DNA encoding one or more cellodextrin transporters and recombinant DNA encoding one or more polypeptides having glucose mutarotase activity, wherein the host cell consumes more molecules of cellobiose when grown in a cellobiose-containing medium than are consumed by a corresponding host cell lacking said recombinant DNA encoding one or more polypeptides having glucose mutarotase activity, wherein the host cell further comprises recombinant DNA encoding one or more β-glucosidases.

In another embodiment, the disclosure provides a host cell including recombinant DNA encoding one or more cellodextrin transporters and recombinant DNA encoding one or more polypeptides having glucose mutarotase activity, wherein the host cell consumes more molecules of cellobiose when grown in a cellobiose-containing medium than are consumed by a corresponding host cell lacking said recombinant DNA encoding one or more polypeptides having glucose mutarotase activity, wherein the polypeptide having mutarotase activity is a polypeptide containing an amino acid sequence selected from the group consisting of SEQ ID NOs: 17, 19, 21, and 23.

In another embodiment, the disclosure provides a host cell including recombinant DNA encoding one or more cellodextrin transporters and recombinant DNA encoding one or more polypeptides having glucose mutarotase activity, wherein the host cell consumes more molecules of cellobiose when grown in a cellobiose-containing medium than are consumed by a corresponding host cell lacking said recombinant DNA encoding one or more polypeptides having glucose mutarotase activity, wherein the polypeptide having mutarotase activity is a polypeptide containing an amino acid sequence selected from the group consisting of SEQ ID NOs: 28-32.

In another embodiment, the disclosure provides a method of fermenting a cellobiose-containing mixture, the method including contacting the cellobiose-containing mixture with a host cell including recombinant DNA encoding one or more cellodextrin transporters and recombinant DNA encoding one or more polypeptides having glucose mutarotase activity, wherein the host cell consumes more molecules of cellobiose when grown in a cellobiose-containing medium than are consumed by a corresponding host cell lacking said recombinant DNA encoding one or more polypeptides having glucose mutarotase activity, and incubating the host cell under conditions that support fermentation.

In another embodiment, the disclosure provides a method of fermenting a cellobiose-containing mixture, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased consumption of cellobiose by the cell as compared with a corresponding host cell lacking said recombinant DNA encoding one or more polypeptides having glucose mutarotase activity.

In another embodiment, the disclosure provides a method of fermenting a cellobiose-containing mixture, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased consumption of cellobiose by the cell as compared with a corresponding host cell lacking said recombinant DNA encoding one or more polypeptides having glucose mutarotase activity, wherein the host cell further comprises recombinant DNA encoding one or more β-glucosidases.

In another embodiment, the disclosure provides a method of fermenting a cellobiose-containing mixture, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased consumption of cellobiose by the cell as compared with a corresponding host cell lacking said recombinant DNA encoding one or more polypeptides having glucose mutarotase activity, wherein the polypeptide having mutarotase activity is a polypeptide containing an amino acid sequence selected from the group consisting of SEQ ID NOs: 17, 19, 21, and 23.

In another embodiment, the disclosure provides a method of fermenting a cellobiose-containing mixture, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased consumption of cellobiose by the cell as compared with a corresponding host cell lacking said recombinant DNA encoding one or more polypeptides having glucose mutarotase activity, wherein the polypeptide having mutarotase activity is a polypeptide containing an amino acid sequence selected from the group consisting of SEQ ID NOs: 28-32.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the host cell further comprises recombinant DNA encoding one or more β-glucosidases.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the polypeptide having mutarotase activity is a polypeptide containing an amino acid sequence selected from the group consisting of SEQ ID NOs: 17, 19, 21, and 23.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the polypeptide having mutarotase activity is a polypeptide containing an amino acid sequence selected from the group consisting of SEQ ID NOs: 28-32.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the chemical is an alcohol.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the chemical is an alcohol, wherein the host cell further comprises recombinant DNA encoding one or more β-glucosidases.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the chemical is an alcohol, wherein the polypeptide having mutarotase activity is a polypeptide containing an amino acid sequence selected from the group consisting of SEQ ID NOs: 17, 19, 21, and 23.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the chemical is an alcohol, wherein the polypeptide having mutarotase activity is a polypeptide containing an amino acid sequence selected from the group consisting of SEQ ID NOs: 28-32.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the chemical is an alcohol, and wherein the alcohol is selected from group consisting of: ethanol, n-propanol, n-butanol, iso-butanol, 3-methyl-1-butanol, 2-methyl-1-butanol, 3-methyl-1-pentanol, and octanol.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the chemical is an alcohol, and wherein the alcohol is selected from group consisting of: ethanol, n-propanol, n-butanol, iso-butanol, 3-methyl-1-butanol, 2-methyl-1-butanol, 3-methyl-1-pentanol, and octanol, wherein the host cell further comprises recombinant DNA encoding one or more β-glucosidases.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the chemical is an alcohol, and wherein the alcohol is selected from group consisting of: ethanol, n-propanol, n-butanol, iso-butanol, 3-methyl-1-butanol, 2-methyl-1-butanol, 3-methyl-1-pentanol, and octanol, wherein the polypeptide having mutarotase activity is a polypeptide containing an amino acid sequence selected from the group consisting of SEQ ID NOs: 17, 19, 21, and 23.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the chemical is an alcohol, and wherein the alcohol is selected from group consisting of: ethanol, n-propanol, n-butanol, iso-butanol, 3-methyl-1-butanol, 2-methyl-1-butanol, 3-methyl-1-pentanol, and octanol, wherein the polypeptide having mutarotase activity is a polypeptide containing an amino acid sequence selected from the group consisting of SEQ ID NOs: 28-32.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the chemical is a terpenoid, a polyketide, a fatty acid, a fatty acid derivative, or an organic acid.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the chemical is a terpenoid, a polyketide, a fatty acid, a fatty acid derivative, or an organic acid, wherein the host cell further comprises recombinant DNA encoding one or more β-glucosidases.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the chemical is a terpenoid, a polyketide, a fatty acid, a fatty acid derivative, or an organic acid, wherein the polypeptide having mutarotase activity is a polypeptide containing an amino acid sequence selected from the group consisting of SEQ ID NOs: 17, 19, 21, and 23.

In another embodiment, the disclosure provides a method of increasing production of a chemical, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity, wherein the chemical is a terpenoid, a polyketide, a fatty acid, a fatty acid derivative, or an organic acid, wherein the polypeptide having mutarotase activity is a polypeptide containing an amino acid sequence selected from the group consisting of SEQ ID NOs: 28-32.

In another embodiment, the disclosure provides a method of increasing the growth rate of a cell, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased growth rate of the cell as compared with a corresponding host cell lacking said recombinant DNA encoding one or more polypeptides having glucose mutarotase activity.

In another embodiment, the disclosure provides a method of increasing the growth rate of a cell, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased growth rate of the cell as compared with a corresponding host cell lacking said recombinant DNA encoding one or more polypeptides having glucose mutarotase activity, wherein the host cell further comprises recombinant DNA encoding one or more β-glucosidases.

In another embodiment, the disclosure provides a method of increasing the growth rate of a cell, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased growth rate of the cell as compared with a corresponding host cell lacking said recombinant DNA encoding one or more polypeptides having glucose mutarotase activity, wherein the polypeptide having mutarotase activity is a polypeptide containing an amino acid sequence selected from the group consisting of SEQ ID NOs: 17, 19, 21, and 23.

In another embodiment, the disclosure provides a method of increasing the growth rate of a cell, the method including providing a host cell, wherein the host cell comprises recombinant DNA encoding one or more cellodextrin transporters and recombinant DNA encoding one or more polypeptides having glucose mutarotase activity, and culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased growth rate of the cell as compared with a corresponding host cell lacking said recombinant DNA encoding one or more polypeptides having glucose mutarotase activity, wherein the polypeptide having mutarotase activity is a polypeptide containing an amino acid sequence selected from the group consisting of SEQ ID NOs: 28-32.

A method of fermenting a β-D-glucose-containing mixture, the method including contacting the β-D-glucose-containing mixture with one or more recombinant polypeptides having glucose mutarotase activity, contacting the β-D-glucose-containing mixture with a cell, wherein the β-D-glucose-containing mixture is contacted with a cell concomitant with or after contacting the β-D-glucose-containing mixture with one or more recombinant polypeptides having glucose mutarotase activity, and incubating the cell and β-D-glucose-containing mixture under conditions that support fermentation, and, wherein contacting the β-D-glucose-containing mixture with the one or more recombinant polypeptides having glucose mutarotase activity results in increased consumption of the β-D-glucose-containing mixture by the cell during fermentation as compared to consumption by the cell of the β-D-glucose-containing mixture not contacted with the one or more recombinant polypeptides having glucose mutarotase activity.

A method of fermenting a β-D-glucose-containing mixture, the method including contacting the β-D-glucose-containing mixture with one or more recombinant polypeptides having glucose mutarotase activity, contacting the β-D-glucose-containing mixture with a cell, wherein the β-D-glucose-containing mixture is contacted with a cell concomitant with or after contacting the β-D-glucose-containing mixture with one or more recombinant polypeptides having glucose mutarotase activity, and incubating the cell and β-D-glucose-containing mixture under conditions that support fermentation, and, wherein contacting the β-D-glucose-containing mixture with the one or more recombinant polypeptides having glucose mutarotase activity results in increased consumption of the β-D-glucose-containing mixture by the cell during fermentation as compared to consumption by the cell of the β-D-glucose-containing mixture not contacted with the one or more recombinant polypeptides having glucose mutarotase activity, wherein the β-D-glucose-containing mixture is obtained from the hydrolysis of cellulose.

A method of fermenting a β-D-glucose-containing mixture, the method including contacting the β-D-glucose-containing mixture with one or more recombinant polypeptides having glucose mutarotase activity, contacting the β-D-glucose-containing mixture with a cell, wherein the β-D-glucose-containing mixture is contacted with a cell concomitant with or after contacting the β-D-glucose-containing mixture with one or more recombinant polypeptides having glucose mutarotase activity, and incubating the cell and β-D-glucose-containing mixture under conditions that support fermentation, and, wherein contacting the β-D-glucose-containing mixture with the one or more recombinant polypeptides having glucose mutarotase activity results in increased consumption of the β-D-glucose-containing mixture by the cell during fermentation as compared to consumption by the cell of the β-D-glucose-containing mixture not contacted with the one or more recombinant polypeptides having glucose mutarotase activity, wherein the polypeptide having mutarotase activity is a polypeptide containing an amino acid sequence selected from the group consisting of SEQ ID NOs: 17, 19, 21, and 23.

A method of fermenting a β-D-glucose-containing mixture, the method including contacting the β-D-glucose-containing mixture with one or more recombinant polypeptides having glucose mutarotase activity, contacting the β-D-glucose-containing mixture with a cell, wherein the β-D-glucose-containing mixture is contacted with a cell concomitant with or after contacting the β-D-glucose-containing mixture with one or more recombinant polypeptides having glucose mutarotase activity, and incubating the cell and β-D-glucose-containing mixture under conditions that support fermentation, and, wherein contacting the β-D-glucose-containing mixture with the one or more recombinant polypeptides having glucose mutarotase activity results in increased consumption of the β-D-glucose-containing mixture by the cell during fermentation as compared to consumption by the cell of the β-D-glucose-containing mixture not contacted with the one or more recombinant polypeptides having glucose mutarotase activity, wherein the polypeptide having mutarotase activity is a polypeptide containing an amino acid sequence selected from the group consisting of SEQ ID NOs: 28-32.

A method of fermenting a β-D-glucose-containing mixture, the method including providing a host cell, wherein the host cell comprises recombinant DNA encoding one or more polypeptides having glucose mutarotase activity, and culturing the host cell in a medium such that the recombinant DNA encoding the one or more polypeptides having glucose mutarotase activity is expressed, and wherein expression of the recombinant DNA encoding one or more polypeptides having glucose mutarotase activity results in increased consumption of β-D-glucose-containing mixture by the cell as compared with a corresponding host cell lacking said recombinant DNA encoding one or more polypeptides having glucose mutarotase activity.

A method of fermenting a β-D-glucose-containing mixture, the method including providing a host cell, wherein the host cell comprises recombinant DNA encoding one or more polypeptides having glucose mutarotase activity, and culturing the host cell in a medium such that the recombinant DNA encoding the one or more polypeptides having glucose mutarotase activity is expressed, and wherein expression of the recombinant DNA encoding one or more polypeptides having glucose mutarotase activity results in increased consumption of β-D-glucose-containing mixture by the cell as compared with a corresponding host cell lacking said recombinant DNA encoding one or more polypeptides having glucose mutarotase activity, wherein the β-D-glucose-containing mixture is obtained from the hydrolysis of cellulose.

A method of fermenting a β-D-glucose-containing mixture, the method including providing a host cell, wherein the host cell comprises recombinant DNA encoding one or more polypeptides having glucose mutarotase activity, and culturing the host cell in a medium such that the recombinant DNA encoding the one or more polypeptides having glucose mutarotase activity is expressed, and wherein expression of the recombinant DNA encoding one or more polypeptides having glucose mutarotase activity results in increased consumption of β-D-glucose-containing mixture by the cell as compared with a corresponding host cell lacking said recombinant DNA encoding one or more polypeptides having glucose mutarotase activity, wherein the polypeptide having mutarotase activity is a polypeptide containing an amino acid sequence selected from the group consisting of SEQ ID NOs: 17, 19, 21, and 23.

A method of fermenting a β-D-glucose-containing mixture, the method including providing a host cell, wherein the host cell comprises recombinant DNA encoding one or more polypeptides having glucose mutarotase activity, and culturing the host cell in a medium such that the recombinant DNA encoding the one or more polypeptides having glucose mutarotase activity is expressed, and wherein expression of the recombinant DNA encoding one or more polypeptides having glucose mutarotase activity results in increased consumption of β-D-glucose-containing mixture by the cell as compared with a corresponding host cell lacking said recombinant DNA encoding one or more polypeptides having glucose mutarotase activity, wherein the polypeptide having mutarotase activity is a polypeptide containing an amino acid sequence selected from the group consisting of SEQ ID NOs: 28-32.

In another embodiment, the disclosure provides a host cell containing recombinant DNA encoding one or more cellodextrin transporters and recombinant DNA encoding one or more polypeptides having glucose mutarotase activity, wherein the host cell consumes more molecules of cellobiose when grown in a cellobiose-containing medium than are consumed by a corresponding host cell lacking said recombinant DNA encoding one or more polypeptides having glucose mutarotase activity, and wherein the polypeptide having glucose mutarotase activity contains one or both amino acid sequences of SEQ ID NO: 28 and 29. In some aspects, the polypeptide having glucose mutarotase activity contains the amino sequences of SEQ ID NOs: 28 and 29. In some of aspects, the host cell further contains recombinant DNA encoding one or more β-glucosidases.

### BRIEF DESCRIPTION OF THE DRAWINGS

FIG. 1 shows the effects of overexpression of five different aldose 1-epimerases on cellobiose utilization in engineered S. cerevisiae parent strain D452-BT. The engineered S. cerevisiae express a recombinant cellodextrin transporter, a β-glucosidase, and an aldose 1-epimerase gene in the multi-copy plasmid pRS423, as indicated by the figure legend. The aldose 1-epimerase genes and source organism are: galM (E. coli); GAL10-Sc (S. cerevisiae, systematic name YBR019C); GAL10-Ps (Pichia stipitis); YHR210C (S. cerevisiae, systematic name YHR210C); YNR071C (S. cerevisiae, systematic name YNR071C). The control organism expressed a recombinant cellodextrin transporter and a β-glucosidase, and contained an empty pRS423 plasmid. Media containing cellobiose was fermented by the S. cerevisiae overexpressing the different aldose 1-epimerases, and cellobiose consumption (left panel; measured in grams of cellobiose per liter), cell growth (middle panel; measured by optical density at 600 nm), and ethanol production (right panel; measured in grams of ethanol per liter) were measured for each strain.

FIG. 2 shows a comparison of ethanol yield, ethanol productivity, and ethanol concentration of during fermentation of media containing cellobiose by S. cerevisiae expressing a recombinant cellodextrin transporter, a β-glucosidase, and an aldose 1-epimerase gene in the multi-copy plasmid pRS423, as indicated by the figure legend. The aldose 1-epimerase genes and source organism are: GAL10-Sc (S. cerevisiae, systematic name YBR019C); YHR210C (S. cerevisiae, systematic name YHR210C); and YNR071C (S. cerevisiae, systematic name YNR071C).

FIG. 3 shows the effects of overexpression of GAL10/YBR019C in S. cerevisiae on the fermentation of cellobiose by S. cerevisiae. A medium containing cellobiose was fermented by control S. cerevisiae expressing a recombinant cellodextrin transporter and β-glucosidase or S. cerevisiae overexpressing GAL10/YBR019C, recombinant cellodextrin transporter, and β-glucosidase and the cell growth (left panel; measured by optical density at 600 nm); cellobiose consumption (middle panel; measured in grams of cellobiose per liter), and ethanol production (right panel; measured in grams of ethanol per liter) were measured for both strains.

FIG. 4 shows the effects of overexpression of two different aldose 1-epimerases on cellobiose utilization in engineered S. cerevisiae parent strain SL01. The engineered S. cerevisiae express a recombinant cellodextrin transporter, a β-glucosidase, and an aldose 1-epimerase gene in the multi-copy plasmid pRS424, as indicated by the figure legend. The aldose 1-epimerase genes and source organism are: scAEP (S. cerevisiae, systematic name YHR210C) and ncAEP (Neurospora crassa, systematic name NCU09705). The control organism expressed a recombinant cellodextrin transporter and a β-glucosidase, and contained an empty pRS424 plasmid. Media containing cellobiose was fermented by the S. cerevisiae overexpressing the different aldose 1-epimerases, and cell growth (top left panel; measured by optical density at 600 nm), cellobiose consumption (top right panel; measured in grams of cellobiose per liter), glucose concentration (bottom left panel; measured in grams of glucose per liter), and ethanol production (bottom right panel; measured in grams of ethanol per liter) were measured for each strain.

FIG. 5 shows the effect of knocking out two aldose 1-epimerases genes from S. cerevisiae on cellobiose utilization by S. cerevisiae. S. cerevisiae contains three putative aldose 1-epimerase genes: YBR019C, YHR210C, and YNR071C. S. cerevisiae SL01 strains with both YHR210C and YNR071C and with both YBR019C and YHR210C genes knocked out were prepared. The different strains as indicated by the figure legend are: “Δ(YHR+YNR)” (YHR210C and YNR071C knockout), “Δ(YHR+GAL10)” (YHR210C and YBR019C knockout), and “control” (no aldose 1-epimerase knocked out). Media containing cellobiose was fermented by the S. cerevisiae aldose 1-epimerase knockout strains, and the cell growth (top panel; measured by optical density at 600 nm); cellobiose consumption (middle panel; measured in grams of cellobiose per liter), and ethanol production (bottom panel; measured in grams of ethanol per liter) were measured for each strain.

FIG. 6 shows the effect of knocking out one aldose 1-epimerase genes from S. cerevisiae on cellobiose utilization by S. cerevisiae. S. cerevisiae contains three putative aldose 1-epimerase genes: YBR019C, YHR210C, and YNR071C. S. cerevisiae SL01 strains with each of the putative aldose 1-epimerase genes knocked were prepared. The different strains as indicated by the figure legend are: ΔYHR (YHR210C knockout), ΔGAL10 (YBR019C knockout), ΔYNR (YNR071C knockout) and “control” (no aldose 1-epimerase knocked out). Media containing cellobiose was fermented by the S. cerevisiae aldose 1-epimerase knockout strains, and the cell growth (top panel; measured by optical density at 600 nm); cellobiose consumption (middle panel; measured in grams of cellobiose per liter), and ethanol production (bottom panel; measured in grams of ethanol per liter) were measured for each strain.

FIG. 7 shows a comparison of glucose fermentation (A) and cellobiose fermentation (B) by an engineered S. cerevisiae D452-BT strain containing both cellobiose transporter (cdt-1) and intracellular β-glucosidase (gh1-1) genes. Symbols: OD (◯), glucose (▾), cellobiose (▴), and ethanol (♦).

FIG. 8 shows a comparison of transcriptomic analysis of AEP by N. crassa in sucrose or Miscanthus hydrolyzate containing medium. 1, 16 h in sucrose; 2, 16 h in Miscanthus; 3, 40 h in Miscanthus; 4, 112 h in Miscanthus; 5, 232 h in Miscanthus.

FIG. 9 shows amino acid sequence alignments of two AEPs from N. crassa and two putative AEPs from S. cerevisiae.

FIG. 10 shows a comparison of cellobiose fermentation by BY4741 ΔYHR, ΔYNR and ΔGAL strains containing a cellobiose fermentation pathway. Error is within 15%. Symbols: control (), ΔYHR (▴), ΔYNR (▪), and ΔGAL (♦).

FIG. 11 shows specific AEP activity of the BY4741 AEP knock-out strains grown up in cellobiose (A) or glucose (B). One unit of AEP activity is defined as the amount of enzyme converting 1 μmol of α-glucose to β-glucose in 1 min in addition to the non-enzymatic rate under 22° C. Error is within 15%.

FIG. 12 shows a comparison of cellobiose fermentation by three S. cerevisiae D452-BT strains overexpressing an AEP gene (GAL10-Sc, YHR210C, or YNR071C) in an engineered S. cerevisiae D452-BT containing a cellobiose fermentation pathway. Symbols: control (), YHR210C (▴), YNR071C (▪), and GAL10-Sc (♦). In all fermentation results, values are the mean of two independent fermentations, and error bars represent the standard deviations.

### DETAILED DESCRIPTION OF PREFERRED EMBODIMENTS

The present disclosure relates to polypeptides having glucose mutarotase activity and methods of their use, nucleotides encoding polypeptides having glucose mutarotase activity, compositions including polypeptides having glucose mutarotase activity and methods of their use, and compositions including nucleic acids encoding polypeptides having glucose mutarotase activity and methods of their use. The disclosure further relates to methods of fermenting cellodextrin-containing materials, methods of increasing consumption of cellodextrins during fermentation of cellodextrin-containing materials, methods of increasing chemical production during fermentation of cellodextrin-containing materials, methods of increasing cell growth during fermentation of cellodextrin-containing materials, and compositions for performing the methods. The disclosure further relates to methods of fermenting β-D-glucose-containing materials, methods of increasing consumption of β-D-glucose during fermentation of β-D-glucose-containing materials, methods of increasing chemical production during fermentation of β-D-glucose-containing materials, methods of increasing cell growth during fermentation of β-D-glucose-containing materials, and compositions for performing the methods.

Cellulose and cellodextrins are composed of β(1-4) linked D-glucose molecules. Hydrolysis of cellodextrins or cellulose into individual glucose molecules results in the release of β-D-glucose molecules. For glucose molecules to be utilized by organisms such as S. cerevisiae for various metabolic pathways, glucose molecules are typically first phosphorylated by a hexokinase. In S. cerevisiae, hexokinases preferably or exclusively phosphorylate α-D-glucose molecules. α-D-glucose molecules may be generated from β-D-glucose molecules by the action of mutarotases, which interconvert the alpha and beta forms of D-glucose.

S. cerevisiae's preference for utilizing α-D-glucose over β-D-glucose is a potentially rate-limiting step in the conversion of cellulose, cellodextrins, and β-D-glucose molecules into useful fermentation chemical products, such as alcohols. Accordingly, provided herein are compositions and methods for converting β-D-glucose molecules into α-D-glucose molecules. Without wishing to be bound by theory, by promoting the conversion of β-D-glucose molecules into α-D-glucose molecules, utilization by S. cerevisiae of β-D-glucose molecules and materials containing β-D-glucose molecules, such as cellodextrins and cellulose, may be increased. Further provided herein are compositions and methods for improved utilization of β-D-glucose and cellodextrins by S. cerevisiae. In addition, the compositions and methods disclosed herein are further applicable to utilization of β-D-glucose or other sugar molecules by organisms that preferentially utilize either an alpha or beta form of a sugar.

As used herein, “aldose 1-epimerase” refers to any polypeptide having glucose mutarotase activity, as defined below. As used herein, “aldose 1-epimerase” also refers to a polynucleotide that encodes an aldose 1-epimerase polypeptide.

As used herein, cellodextrin refers to glucose polymers of varying length and includes, without limitation, cellobiose (2 glucose monomers), cellotriose (3 glucose monomers), cellotetraose (4 glucose monomers), cellopentaose (5 glucose monomers), and cellohexaose (6 glucose monomers).

As used herein, sugar refers to monosaccharides (e.g., glucose, fructose, galactose, xylose, arabinose), disaccharides (e.g., cellobiose, sucrose, lactose, maltose), and oligosaccharides (typically containing 3 to 10 component monosaccharides).

### Polypeptides of the Disclosure

As used herein, a “polypeptide” is an amino acid sequence including a plurality of consecutive polymerized amino acid residues (e.g., at least about 15 consecutive polymerized amino acid residues). The polypeptide optionally comprises modified amino acid residues, naturally occurring amino acid residues not encoded by a codon, and non-naturally occurring amino acid residues.

As used herein, “protein” refers to an amino acid sequence, oligopeptide, peptide, polypeptide, or portions thereof whether naturally occurring or synthetic.

Polypeptides Having Glucose Mutarotase Activity

Disclosed herein are recombinant polypeptides having glucose mutarotase activity. As used herein, “mutarotase activity” refers to the ability of an enzyme to convert 3-D-glucose to α-D-glucose and/or to convert α-D-glucose to β-D-glucose. “Mutarotase activity” may also refer to the ability of an enzyme to convert between the alpha and beta forms of other sugars, including L-arabinose, D-xylose, D-galactose, maltose and lactose.

Recombinant polypeptides having glucose mutarotase activity disclosed herein include, without limitation, the S. cerevisiae polypeptides GAL10/YBR019C (SEQ ID NO: 17), YHR210C (SEQ ID NO: 19), and YNR071C (SEQ ID NO: 21) and the N. crassa polypeptide NCU09705 (SEQ ID NO: 23).

In one embodiment, a polypeptide having glucose mutarotase activity is the S. cerevisiae polypeptide GAL10/YBR019C. The S. cerevisiae GAL10 polypeptide is referred to by a variety of names in the literature, including aldose 1-epimerase, UDP-glucose 4-epimerase, UDP-galactose 4-epimerase, and mutarotase. S. cerevisiae GAL10 is a bi-functional enzyme, where the N-terminal portion of the protein has UDP-glucose epimerase activity (converting between UDP-glucose and UDP-galactose), and the C-terminal portion of the protein has mutarotase activity. The crystal structure of the S. cerevisiae GAL10 enzyme is disclosed in a recent journal article (see Thoden and Holden, (2005) J Biol Chem 280 (23): 21900-21907).

In one aspect, a polypeptide having glucose mutarotase activity is the full-length GAL10/YBR019C protein (SEQ ID NO: 17). In another aspect, a polypeptide having glucose mutarotase activity is a polypeptide having about the C-terminal half of the GAL10/YBR019C protein. In another aspect, a polypeptide having glucose mutarotase activity is amino acid residues Ile-378 to Ser-699 of the GAL10/YBR019C protein (SEQ ID NO: 30) (amino acid numbers 378-699 of SEQ ID NO: 17). In another aspect, a polypeptide having glucose mutarotase activity is amino acid residues Glu-361 to Ser-699 of the GAL10/YBR019C protein (SEQ ID NO: 31) (amino acid numbers 361-699 of SEQ ID NO: 17). In another aspect, a polypeptide having glucose mutarotase activity is amino acid residues Phe-364 to Ser-699 of the GAL10/YBR019C protein (SEQ ID NO: 32) (amino acid numbers 364-699 of SEQ ID NO: 17). As would be understood by one having skill in the art, additional truncated versions of the GAL10/YBR019C can be prepared, in which N-terminal amino acids involved primarily or entirely in the epimerase activity of GAL10/YBR019C are removed, and the C-terminal amino acids required for mutarotase activity are preserved. Such truncated versions of GAL10/YBR019C may be identified, for example, by preparing various truncated versions of GAL10/YBR019C protein, and testing the truncated proteins for glucose mutarotase enzymatic activity. Methods for the preparation of truncated versions proteins are well known in the art, and may involved, for example, generating truncated versions of a gene encoding GAL10/YBR019C protein through PCR, cloning the gene encoding a truncated version of the protein into an expression vector, transforming a host cell with the expression vector, and expressing the protein in the host cell.

In another embodiment, a polypeptide having glucose mutarotase activity is the S. cerevisiae polypeptide YHR210C (SEQ ID NO: 19). In one aspect, the YHR210C polypeptide sequence may be aligned based on sequence homology with amino acid residues Phe-364 to Ser-699 of the GAL10/YBR019C protein.

In another embodiment, a polypeptide having glucose mutarotase activity is the S. cerevisiae polypeptide YNR071C (SEQ ID NO: 21). In one aspect, the YNR071C polypeptide sequence may be aligned based on sequence homology with amino acid residues Phe-364 to Ser-699 of the GAL10/YBR019C protein.

In another embodiment, a polypeptide having glucose mutarotase activity is the Neurospora crassa polypeptide NCU09705 (SEQ ID NO: 23). In one aspect, the NCU09705 polypeptide sequence may be aligned based on sequence homology with amino acid residues Ala-386 to Arg-697 of the GAL10/YBR019C protein.

In another embodiment, a polypeptide having glucose mutarose activity is a polypeptide that contains one or both of the following amino acid motifs:

Motif 1: G-X-[VTI]-[VPI]-G-R-[VTY]-[AT]-N-R-[VILT] (SEQ ID NO: 28) (corresponds to residues 424-434 of GAL/YBR019C), wherein X is any amino acid, and, for an amino acid position in brackets, the amino acid is any of the bracketed amino acids. For example, [VTI] is V, T, or I.

Motif 2: T-[VPI][VI]MGNFX-[STA]NSQHP1-H-[IST]-Y-[FW]-N-L (SEQ ID NO: 29) (corresponds to residues 530-542 of GAL/YBR019C), wherein X is any amino acid and, for an amino acid position in brackets, the amino acid is any of the bracketed amino acids. For example, [VPI] is V, P, or I.

In one aspect, a polypeptide having glucose mutarotase activity has an amino acid sequence containing the amino acid sequence of SEQ ID NO: 28. In one aspect, a polypeptide having glucose mutarotase activity has an amino acid sequence containing the amino acid sequence of SEQ ID NO: 29. In one aspect, a polypeptide having glucose mutarotase activity has an amino acid sequence containing the amino acid sequences of SEQ ID NO: 28 and SEQ ID NO: 29 above.

In certain embodiments, polypeptides having glucose mutarotase activity are polypeptides having at least about 20%, or at least about 29%, or at least about 30%, or at least about 40%, or at least about 50%, or at least about 55%, or at least about 60%, or at least about 65%, or at least about 70%, or at least about 75%, or at least about 80%, or at least about 85%, or at least about 90%, or at least about 92%, or at least about 94%, or at least about 96%, or at least about 98%, or at least about 99%, or 100% amino acid residue sequence identity to the polypeptide of GAL10/YBR019C, YHR210C, YNR071C or NCU09705. In certain embodiments, polypeptides having glucose mutarotase activity are polypeptides having at least 7, 8, 9, 10, 11, 12, 13, 14, 15, 20, 25, 30, 35, 40, 45, or 50 consecutive amino acids of the polypeptides of GAL10/YBR019C, YHR210C, YNR071C or NCU09705.

Polypeptides having glucose mutarotase activity further include recombinant polypeptides that are conservatively modified variants of polypeptides of GAL10/YBR019C, YHR210C, and YNR071C, and NCU09705. “Conservatively modified variants” as used herein include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain examples of amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Recombinant polypeptides having glucose mutarotase activity further include polypeptides that are homologs or orthologs of polypeptides GAL10/YBR019C, YHR210C, YNR071C and NCU09705. “Homology” as used herein refers to sequence identity between a reference sequence and at least a fragment of a second sequence. Homologs may be identified by any method known in the art, preferably, by using the BLAST tool to compare a reference sequence to a single second sequence or fragment of a sequence or to a database of sequences. As described below, BLAST will compare sequences based upon percent identity and similarity. “Orthology” as used herein refers to genes in different species that derive from a common ancestor gene.

### Polynucleotides of the Disclosure

As used herein, the terms “polynucleotide,” “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; inter-nucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature.

Polynucleotides Encoding Polypeptides Having Mutarotase Activity

The present disclosure includes recombinant polynucleotides encoding polypeptides having mutarotase activity. The disclosure further relates to host cells and methods of using such host cells where the host cells comprise recombinant polynucleotides encoding polypeptides having glucose mutarotase activity.

Recombinant polynucleotides of the disclosure include any polynucleotide that encodes a polypeptide as disclosed herein having glucose mutarotase activity. In some aspects, polynucleotides of the disclosure include polynucleotides that encode a polypeptide of SEQ ID NO: 17 (GAL10/YBR019C polypeptide), SEQ ID NO: 19 (YHR210C polypeptide), SEQ ID NO: 21 (YNR071C polypeptide), SEQ ID NO: 23 (NCU09705 polypeptide). In some aspects, polynucleotides of the disclosure include the polynucleotides of: SEQ ID NO: 16 (encodes GAL10/YBR019C polypeptide), SEQ ID NO: 18 (encodes YHR210C polypeptide), SEQ ID NO: 20 (encodes YNR071C polypeptide), and SEQ ID NO: 22 (encodes NCU09705 polypeptide).

In certain aspects, the recombinant polynucleotides of the disclosure include polynucleotides having at least about 20%, at least about 30%, at least about 40%, at least about 50%, at least about 55%, at least about 60%, at least about 65%, at least about 70%, at least about 75%, at least about 80%, at least about 85%, at least about 90%, at least about 92%, at least about 94%, at least about 96%, at least about 98%, at least about 99%, or 100% nucleotide residue sequence identity to the polynucleotide of SEQ ID NO: 16, SEQ ID NO: 18, SEQ ID NO: 20, and SEQ ID NO: 22.

Polynucleotides of the disclosure also include polynucleotides that encode a polypeptide having one or both of the amino acid sequences of SEQ ID NO: 28 and SEQ ID NO: 29.

Polynucleotides of the disclosure further include polynucleotides that encode conservatively modified variants of polypeptides of GAL10/YBR019C, YHR210C, YNR071C, and NCU09705. Polynucleotides of the disclosure further include polynucleotides that encode homologs or orthologs of polypeptides of GAL10/YBR019C, YHR210C, YNR071C, and NCU09705.

Sequences of the polynucleotides of the disclosure are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature [e.g., in Matteucci et al., (1980) Tetrahedron Lett 21:719-722; U.S. Pat. Nos. 4,500,707; 5,436,327; and 5,700,637]. In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each polynucleotide of the disclosure can be incorporated into an expression vector. “Expression vector” or “vector” refers to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present disclosure include those into which a nucleic acid sequence can be inserted, along with any preferred or required operational elements. Further, the expression vector must be one that can be transferred into a host cell and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

Incorporation of the individual polynucleotides may be accomplished through known methods that include, for example, the use of restriction enzymes (such as BamHI, EcoRI, HhaI, XhoI, XmaI, and so forth) to cleave specific sites in the expression vector, e.g., plasmid. The restriction enzyme produces single stranded ends that may be annealed to a polynucleotide having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired polynucleotide are often cleaved with the same restriction enzyme, thereby assuring that the ends of the expression vector and the ends of the polynucleotide are complementary to each other. In addition, DNA linkers maybe used to facilitate linking of nucleic acids sequences into an expression vector.

A series of individual polynucleotides can also be combined by utilizing methods that are known to those having ordinary skill in the art (e.g., U.S. Pat. No. 4,683,195). For example, each of the desired polynucleotides can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other. Extension of this overlap by DNA polymerase produces a molecule in which the original sequences are “spliced” together. In this way, a series of individual polynucleotides may be “spliced” together and subsequently transduced into a host cell simultaneously. Thus, expression of each of the plurality of polynucleotides is affected.

Individual polynucleotides, or “spliced” polynucleotides, are then incorporated into an expression vector. The disclosure is not limited with respect to the process by which the polynucleotide is incorporated into the expression vector. Those of ordinary skill in the art are familiar with the necessary steps for incorporating a polynucleotide into an expression vector. A typical expression vector contains the desired polynucleotide preceded by one or more regulatory regions, along with a ribosome binding site, e.g., a nucleotide sequence that is 3-9 nucleotides in length and located 3-11 nucleotides upstream of the initiation codon in E. coli. See Shine and Dalgarno (1975) Nature 254(5495):34-38 and Steitz (1979) Biological Regulation and Development (ed. Goldberger, R. F.), 1:349-399 (Plenum, New York).

The term “operably linked” as used herein refers to a configuration in which a control sequence is placed at an appropriate position relative to the coding sequence of the DNA sequence or polynucleotide such that the control sequence directs the expression of a polypeptide.

Regulatory regions include, for example, those regions that contain a promoter and an operator. A promoter is operably linked to the desired polynucleotide, thereby initiating transcription of the polynucleotide via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. Examples include lactose promoters (Lad repressor protein changes conformation when contacted with lactose, thereby preventing the Lad repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Another example is the tac promoter (see de Boer et al., (1983) Proc Natl Acad Sci USA 80(1):21-25). As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M13 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

### Sequence Alignment and Sequence Identity

Methods of alignment of sequences for comparison are well-known in the art. For example, the determination of percent sequence identity between any two sequences can be accomplished using a mathematical algorithm. Non-limiting examples of such mathematical algorithms are the algorithm of Myers and Miller (1988) CABIOS 4:11 17; the local homology algorithm of Smith et al. (1981) Adv. Appl. Math. 2:482; the homology alignment algorithm of Needleman and Wunsch (1970) J. Mol. Biol. 48:443 453; the search-for-similarity-method of Pearson and Lipman (1988) Proc. Natl. Acad. Sci. 85:2444 2448; the algorithm of Karlin and Altschul (1990) Proc. Natl. Acad. Sci. USA 872264, modified as in Karlin and Altschul (1993) Proc. Natl. Acad. Sci. USA 90:5873 5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237 244 (1988); Higgins et al. (1989) CABIOS 5:151 153; Corpet et al. (1988) Nucleic Acids Res. 16:10881 90; Huang et al. (1992) CABIOS 8:155 65; and Pearson et al. (1994) Meth. Mol. Biol. 24:307 331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

As used herein, sequence identity or identity in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical and often differ by conservative amino acid substitutions, where amino acid residues are substituted for other amino acid residues with similar chemical properties (e.g., charge or hydrophobicity), do not change the functional properties of the molecule. When sequences differ in conservative substitutions, the percent sequence identity may be adjusted upwards to correct for the conservative nature of the substitution. Sequences that differ by such conservative substitutions are said to have sequence similarity or similarity. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

Other than percentage of sequence identity noted above, another indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

### Compositions Including Polypeptides Having Glucose Mutarotase Activity

The disclosure further provides compositions including polypeptides having glucose mutarotase activity. In one aspect, recombinant polypeptides having glucose mutarotase activity are provided. In one aspect, a host cell including one or more polypeptides having glucose mutarotase activity is provided.

### Recombinant Polypeptides Having Glucose Mutarotase Activity

In some aspects, recombinant polypeptides having glucose mutarotase activity are provided. In some aspects, recombinant polypeptides having glucose mutarotase activity include the S. cerevisiae polypeptides GAL10/YBR019C, YHR210C, and YNR071C and the N. crassa polypeptide NCU09705, and variants thereof, as described supra. In some aspects, recombinant polypeptides having glucose mutarotase activity include polypeptides containing the amino acid sequence of one or both of SEQ ID NO: 28 and 29.

Recombinant polypeptides having glucose mutarotase activity may be prepared by standard molecular biology techniques such as those described in Sambrook, J. et al. 2000 Molecular Cloning: A Laboratory Manual (Third Edition). Recombinant polypeptides may be expressed in and purified from transgenic expression systems. Transgenic expression systems can be prokaryotic or eukaryotic. Transgenic hosts cells may include yeast and E. coli. In some aspects, transgenic host cells may secrete the polypeptide out of the host cell. In some aspects, transgenic host cells may retain the expressed polypeptide in the host cell.

In certain aspects, recombinant polypeptides having glucose mutarotase activity are isolated from a host cell. In certain aspects, a recombinant polypeptide having glucose mutarotase activity is prepared with a protein “tag” to facilitate protein purification, such as a GST-tag or poly-His tag. In some aspects, recombinant polypeptides having glucose mutarotase activity may be purified to a high degree of purity (e.g. >99% pure, >98% pure, >95% pure, >90% pure, etc.). Recombinant polypeptides may be purified through a variety of techniques known to those of skill in the art, including for example, ion-exchange chromatography, size exclusion chromatography, and affinity chromatography.

### Host Cells of the Disclosure

The disclosure herein relates to host cells containing recombinant polynucleotides encoding polypeptides having glucose mutarotase activity.

“Host cell” and “host microorganism” are used interchangeably herein to refer to a living biological cell that can be transformed via insertion of recombinant DNA or RNA. Such recombinant DNA or RNA can be in an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

Any prokaryotic or eukaryotic host cell may be used in the present disclosure so long as it remains viable after being transformed with a sequence of nucleic acids. Preferably, the host cell is not adversely affected by the transduction of the necessary nucleic acid sequences, the subsequent expression of the proteins (e.g., transporters), or the resulting intermediates. Suitable eukaryotic cells include, but are not limited to, fungal, plant, insect or mammalian cells.

In certain embodiments, the host is a fungal strain. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In certain embodiments, the fungal host is a yeast strain. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this disclosure, yeast shall be defined as described in Biology and Activities of Yeast (Skinner, F. A., Passmore, S. M., and Davenport, R. R., eds, Soc. App. Bacteriol. Symposium Series No. 9, 1980).

In certain embodiments, the yeast host is a Candida, Hansenula, Kluyveromyces, Pichia, Saccharomyces, Schizosaccharomyces, or Yarrowia strain.

In some embodiments of the invention, the host cell is Saccharomyces sp., Saccharomyces cerevisiae, Saccharomyces monacensis, Saccharomyces bayanus, Saccharomyces pastorianus, Saccharomyces carlsbergensis, Saccharomyces pombe, Kluyveromyces sp., Kluyveromyces marxiamus, Kluyveromyces lactis, Kluyveromyces fragilis, Pichia stipitis, Sporotrichum thermophile, Candida shehatae, Candida tropicalis, Neurospora crassa, Zymomonas mobilis. In other embodiments, the yeast host may be Yarrowia lipolytica, Brettanomyces custersii, or Zygosaccharomyces roux.

Saccharomyces sp. may include industrial Saccharomyces strains. Argueso et al. discuss the genome structure of an Industrial Saccharomyces strain commonly used in bioethanol production as well as specific gene polymorphisms that are important for bioethanol production (Argueso et al., Genome Research, 19: 2258-2270, 2009).

In another embodiment, the fungal host is a filamentous fungal strain. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In certain embodiments, the filamentous fungal host is, but not limited to, an Acremonium, Aspergillus, Fusarium, Humicola, Mucor, Myceliophthora, Neurospora, Penicillium, Scytalidium, Thielavia, Tolypocladium, or Trichoderma strain.

In certain embodiments, the filamentous fungal host is an Aspergillus awamori, Aspergillus foetidus, Aspergillus japonicus, Aspergillus nidulans, Aspergillus niger, or Aspergillus oryzae strain. In other embodiments, the filamentous fungal host is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum strain. In yet other embodiments, the filamentous fungal host is a Humicola insolens, Humicola lanuginosa, Mucor miehei, Myceliophthora thermophila, Neurospora crassa, Penicillium purpurogenum, Scytalidium thermophilum, Sporotrichum thermophile, or Thielavia terrestris strain. In a further embodiment, the filamentous fungal host is a Trichoderma harzianum, Trichoderma koningii, Trichoderma longibrachiatum, Trichoderma reesei, or Trichoderma viride strain.

In other embodiments, the host cell is prokaryotic, and in certain embodiments, the prokaryotes are E. coli, Bacillus subtilis, Zymomonas mobilis, Clostridium sp., Clostridium phytofermentans, Clostridium thermocellum, Clostridium beijerinckii, Clostridium acetobutylicum (Moorella thermoacetica), Thermoanaerobacterium saccharolyticum, or Klebsiella oxytoca. In other embodiments, the prokaryotic host cells are Carboxydocella sp., Corynebacterium glutamicum, Enterobacteriaceae, Erwinia chrysanthemi, Lactobacillus sp., Pediococcus acidilactici, Rhodopseudomonas capsulata, Streptococcus lactis, Vibrio furnissii, Vibrio furnissii M1, Caldicellulosiruptor saccharolyticus, or Xanthomonas campestris. In other embodiments, the host cells are cyanobacteria. Additional examples of bacterial host cells include, without limitation, those species assigned to the Escherichia, Enterobacter, Azotobacter, Erwinia, Bacillus, Pseudomonas, Klebsiella, Proteus, Salmonella, Serratia, Shigella, Rhizobia, Vitreoscilla, Synechococcus, Synechocystis, and Paracoccus taxonomical classes.

The host cells of the present disclosure may be genetically modified in that recombinant nucleic acids have been introduced into the host cells, and as such the genetically modified host cells do not occur in nature. The suitable host cell is one capable of expressing one or more nucleic acid constructs encoding one or more proteins for different functions.

“Recombinant nucleic acid” or “heterologous nucleic acid” or “recombinant polynucleotide”, “recombinant nucleotide” or “recombinant DNA” as used herein refers to a polymer of nucleic acids wherein at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids comprises two or more subsequences that are not found in the same relationship to each other in nature. For example, regarding instance (c), a recombinant nucleic acid sequence will have two or more sequences from unrelated genes arranged to make a new functional nucleic acid. Specifically, the present invention describes the introduction of an expression vector into a host cell, wherein the expression vector contains a nucleic acid sequence coding for a protein that is not normally found in a host cell or contains a nucleic acid coding for a protein that is normally found in a cell but is under the control of different regulatory sequences. With reference to the host cell's genome, then, the nucleic acid sequence that codes for the protein is recombinant. As used herein, the term “recombinant polypeptide” refers to a polypeptide generated from a “recombinant nucleic acid” or “heterologous nucleic acid” or “recombinant polynucleotide”, “recombinant nucleotide” or “recombinant DNA” as described above.

In some embodiments, the host cell naturally produces any of the proteins encoded by the polynucleotides of the invention. The genes encoding the desired proteins may be heterologous to the host cell or these genes may be endogenous to the host cell but are operatively linked to heterologous promoters and/or control regions which result in the higher expression of the gene(s) in the host cell. In other embodiments, the host cell does not naturally produce the desired proteins, and comprises heterologous nucleic acid constructs capable of expressing one or more genes necessary for producing those molecules.

### Host Cell Components

In one aspect, host cells of the current disclosure contain recombinant DNA encoding one or more polypeptides having glucose mutarotase activity disclosed herein. In one aspect, host cells of the disclosure overexpress one or more polypeptides having glucose mutarotase activity (i.e. the host cell expresses more of the polypeptide having glucose mutarotase activity than a corresponding host cell lacking recombinant DNA encoding one or more polypeptides having glucose mutarotase activity). In one aspect, host cells of the current disclosure contain recombinant DNA encoding S. cerevisiae GAL10/YBR019C polypeptide. In another aspect, host cells of the current disclosure contain recombinant DNA encoding S. cerevisiae YHR210C polypeptide. In another aspect, host cells of the current disclosure contain recombinant DNA encoding S. cerevisiae YNR071C polypeptide. In another aspect, host cells of the current disclosure contain recombinant DNA encoding N. crassa NCU09705 polypeptide. In another aspect, host cells of the current disclosure contain recombinant DNA encoding a variant or truncated version of S. cerevisiae GAL10/YBR019C, YHR210C, or YNR071C polypeptide, or N. crassa NCU09705 polypeptide. In another aspect, host cells of the current disclosure contain recombinant DNA encoding a polypeptide containing one or both of the amino acid sequences of SEQ ID NO: 28 and SEQ ID NO: 29.

In certain aspects, the polypeptide has at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the GAL10/YBR019C, YHR210C, YNR071C, or NCU09705 polypeptide.

Cellodextrin Transporters

In certain embodiments, the host cell further contains recombinant DNA encoding one or more cellodextrin transporters. A cellodextrin transporter is any transmembrane protein that transports a cellodextrin molecule from outside of the cell to the inside of the cell and/or from inside of the cell to outside of the cell. In certain embodiments, the cellodextrin transporter is a functional fragment that maintains the ability to transport a cellodextrin molecule from outside of the cell to the inside of the cell and/or from inside of the cell to outside of the cell.

Recombinant cellodextrin transporters of the present disclosure may be encoded by any of the genes listed in Table 10, in Supplemental Data, Dataset S1, page 3 in Tian et al., Proc. Natl. Acad. Sci. U.S.A. 106 (52):22157-22162, 2009; and in Tables 1 and 2 provided below.

TABLE 1
Listing of sequences encoding cellodextrin transporters.

| | | NCBI Reference | |
|---|---|---|---|
| Gene | Alternate | Sequence/GenBank | |
| Name/Locus | Name | Accession Number | Organism |
| NCU00801 | cbt1 | XP_963801.1/EAA34565 | N. crassa |
| NCU00809 | | XP_964302.1/EAA35116.1 | N. crassa |
| NCU00821 | AN25 | XP_964364.2/EAA35128.2 | N. crassa |
| NCU00988 | Xy33 | XP_963898.1/EAA34662.1 | N. crassa |
| NCU01231 | | XP_961597.2/EAA32361.2 | N. crassa |
| NCU01494 | AN49 | XP_955927.2/EAA26691.2 | N. crassa |
| NCU02188 | AN28-3 | XP_959582.2/EAA30346.2 | N. crassa |
| NCU04537 | Xy50 | XP_955977.1/EAA26741.1 | N. crassa |
| NCU04963 | AN29-2 | XP_959411.2/EAA30175.2 | N. crassa |
| NCU05519 | | XP_960481.1/EAA31245.1 | N. crassa |
| NCU05853 | | XP_959844.1/EAA30608.1 | N. crassa |
| NCU05897 | | XP_959888.1/EAA30652.1 | N. crassa |
| NCU06138 | Xy31 | XP_960000.1/EAA30764.1 | N. crassa |
| NCU08114 | cbt2 | XP_963873.1/EAA34637.1 | N. crassa |
| NCU09287 | AN41 | XP_958139.1/EAA28903.1 | N. crassa |
| NCU10021 | | XP_958069.2/EAA28833.2 | N. crassa |
| XP_001387242 | Ap26 | XP_001387242 | P. stipitis |
| HGT3 | Xyp30-1 | XP_001386715.1/ABN68686.1 | P. stipitis |
| STL1 | Xyp30 | XP_001383774.1/ABN65745.1 | P. stipitis |
| STL12/XUT6 | Xyp29 | XP_001386589.1/ABN68560.1 | P. stipitis |
| SUT2 | Ap31 | XP_001384295.2/ABN66266.2 | P. stipitis |
| SUT3 | Xyp37 | XP_001386019.2/ABN67990.2 | P. stipitis |
| XUT1 | Xyp32 | XP_001385583.1/ABN67554.1 | P. stipitis |
| XUT2 | Xyp31 | XP_001387242.1/EAZ63219.2 | P. stipitis |
| XUT3 | Xyp33 | XP_001387138.1/EAZ63115.1 | P. stipitis |
| XUT7 | Xyp28 | XP_001387067.1/EAZ63044.1 | P. stipitis |
| NCU07705 | cdr-1 | XP_962291.1/EAA33055 | N. crassa |
| NCU05137 | | XP_956635.1/EAA27399 | N. crassa |
| NCU01517 | | XP_956966.1/EAA27730 | N. crassa |
| NCU09133 | | XP_958905.1/EAA29669 | N. crassa |
| NCU10040 | | | N. crassa |

TABLE 2
Listing of cellodextrin transporter orthologs.

| | | NCBI Reference Sequence/ |
|---|---|---|
| N. crassa | | NCBI GI Number/ |
| ortholog | Organism | JGI number ¥ |
| NCU00809 | Chaetomium globusom CBS148.51 | XP_001220480 |
| NCU00809 | Podospora anserina | XP_001912722 |
| NCU00809 | Nectria haematococca mpVI77-13-4 | EEU41662 |
| NCU00809 | Aspergillus nidulans FGSC A4 | XP_660803 |
| NCU00809 | Aspergillus terreus NIH2624 | XP_001218592 |
| NCU00809 | Talaromyces stipitatus ATCC 10500 | XP_002341594 |
| NCU00809 | Aspergillus niger | XP_001395979 |
| NCU00809 | Aspergillus fumigatus Af293 | XP_747891 |
| NCU00809 | Aspergillus terreus NIH2624 | XP_00120996 |
| NCU00809 | Aspergillus oryzae RIB40 | XP_001817400 |
| NCU08114 | Podospora anserina | XP_001908539 |
| NCU08114 | Penicillium chrysogenum Wisconsin 54-1255 | XP_002568019 |
| NCU08114 | Aspergillus terreus NIH2624 | XP_001209810 |
| NCU08114 | Aspergillus oryzae RIB40 | XP_001820343 |
| NCU08114 | Aspergillus terreus NIH2624 | XP_001210859 |
| NCU08114 | Neurospora crassa OR74A | XP_001728155 |
| NCU08114 | Aspergillus oryzae RIB40 | XP_001826848 |
| NCU08114 | Aspergillus nidulans FGSC A4 | XP_657617 |
| NCU08114 | Talaromyces stipitatus ATCC 10500 | XP_002487579 |
| NCU08114 | Chaetomium globosum CBS 148.51 | XP_001227497 |
| NCU08114 | Trichoderma atroviridae | 215408 |
| NCU08114 | Chaetomium globosum | XP_001220290.1 |
| NCU08114 | Aspergillus nidulans | ANID_08347 |
| NCU08114 | Pleurotus ostreatus | 51322 |
| NCU08114 | Sporotrichum thermophile | 114107 |
| NCU00801 | Aspergillus nidulans | XP_660418.1 |
| NCU00801 | Magnaporthe grisea | XP_364883.1 |
| NCU00801 | Aspergillus fumigatus | XP_753099.1 |
| NCU00801 | Trichoderma atroviridae | 211304 |
| NCU00801 | Chaetomium globosum | XP_001220469.1 |
| NCU00801 | Tremella mesenterica | 63529 |
| NCU00801 | Heterobasidion. annosum | 105952 |
| NCU00801 | Cryphonectria parasitica | 252427 |
| NCU00801 | Trichoderma reesei | 67752 |
| NCU00801 | Aspergillus clavatus | XP_001268541.1 |
| NCU00801 | Neurospora discreta | 77429 |
| NCU00801 | Trichoderma reesei | 3405 |
| NCU00801 | Sporotrichum thermophile | 43941 |
| NCU00801 | Neurospora crassa | XP_963801.1 |
| NCU05853 | Chaetomium globosum | XP_001226269.1 |
| NCU05853 | Trichoderma reesei | 46819 |
| NCU05853 | Mycosphaerella graminicola | 68287 |
| NCU05853 | Aspergillus flavus | AFLA_000820A |
| NCU00809 | Pichia stipitis CBS6054 | XP_001383110.1/GI: 126133170 |
| NCU00809 | Pichia stipitis CBS6054 | XP_001387231.1/GI: 126276337 |
| NCU00809 | Pichia stipitis CBS6054 | XP_001383677.2/GI: 150864727 |
| NCU08114 | Pichia stipitis CBS6054 | XP_001386873.1/GI: 126275571 |
| NCU05853 | Pichia stipitis CBS6054 | XP_001382754.1/GI: 126132458 |
| NCU08114 | Pichia stipitis CBS6054 | XP_001387757.1/GI: 126273939 |
| NCU08114 | Pichia stipitis CBS6054 | XP_001385684.1/GI: 126138322 |
| NCU08114 | Pichia stipitis CBS6054 | XP_001384653.2/GI: 15086543 |

¥ When accession numbers were not available, the JGI number was used. The JGI number allows access to the gene sequence via the JGI genome portal for this organism (accessible from the following page: genome.jgi-psf.org/programs/fungi/index.jsf). The A. flavus and A. nidulans identifiers allow access to the genes through their genome portals at webpage cadre-genomes.org.uk/ and webpage broadinstitute.org/annotation/genome/aspergillus_group/MultiHome.html, respectively.

In other embodiments, a recombinant cellodextrin transporter of the present disclosure has about 20%, at least about 30%, at least about 40%, at least about 50%, at least about 55%, at least about 60%, at least about 65%, at least about 70%, at least about 75%, at least about 80%, at least about 85%, at least about 90%, at least about 92%, at least about 94%, at least about 96%, at least about 98%, at least about 99%, or 100% amino acid residue sequence identity to a polypeptide encoded by any of the genes listed in genes listed in Table 10, in Supplemental Data, Dataset S1, page 3 in Tian et al., 2009, supra; and in Tables 1 and 2.

Additionally, cellodextrin transporters of the present disclosure include, without limitation, NCU00801, NCU00809, NCU08114, XP_{—}001268541.1, LAC2, NCU00130, NCU00821, NCU04963, NCU07705, NCU05137, NCU01517, NCU09133, and NCU10040. In certain embodiments, the recombinant cellodextrin transporter has at least about 20%, at least about 30%, at least about 40%, at least about 50%, at least about 55%, at least about 60%, at least about 65%, at least about 70%, at least about 75%, at least about 80%, at least about 85%, at least about 90%, at least about 92%, at least about 94%, at least about 96%, at least about 98%, at least about 99%, or 100% amino acid residue sequence identity to a polypeptide encoded by any of the sequences NCU00801, NCU00809, NCU08114, XP_{—}001268541.1, LAC2, NCU00130, NCU00821, NCU04963, NCU07705, NCU05137, NCU01517, NCU09133, or NCU10040.

In certain embodiments, the host cell contains a cellodextrin transporter encoded by NCU00801, which is also known as cdt-1 or CBT1. In other embodiments, the host cell contains a cellodextrin transporter encoded by NCU08114, which is also known as Cdt-2 or CBT2. In certain embodiments, the recombinant cellodextrin transporter has an amino acid sequence with at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% amino acid identity to Cdt-1 (SEQ ID NO: 33) or Cdt-2 (SEQ ID NO: 34).

Suitable cellodextrin transporters of the present disclosure also include, without limitation, those described in U.S. Pat. Application Publication No. US 2011/0262983 and PCT Publication No. WO 2011/123715. For example, suitable cellodextrin transporters may include, without limitation, HXT2.1, HXT2.2, HXT2.3, HXT2.4, HXT2.5, HXT2.6, and HXT4. In certain embodiments, a recombinant cellodextrin transporter of the present disclosure has about 20%, at least about 30%, at least about 40%, at least about 50%, at least about 55%, at least about 60%, at least about 65%, at least about 70%, at least about 75%, at least about 80%, at least about 85%, at least about 90%, at least about 92%, at least about 94%, at least about 96%, at least about 98%, at least about 99%, or 100% amino acid residue sequence identity to amino acid residue sequence identity to a polypeptide encoded by any of the genes listed in genes listed in U.S. Pat. Application Publication No. US 2011/0262983 and PCT Publication No. WO 2011/123715 (e.g., HXT2.1, HXT2.2, HXT2.3, HXT2.4, HXT2.5, HXT2.6, or HXT4).

Recombinant cellodextrin transporters of the present disclosure may also include, without limitation, polypeptides encoded by polynucleotides that encode conservatively modified variants of polypeptides encoded by the genes listed above. Recombinant cellodextrin transporters of the present disclosure further include polypeptides encoded by polynucleotides that encode homologs or orthologs of polypeptides encoded by any of the genes listed in Table 10, in Supplemental Data, Dataset S1, page 3 in Tian et al., 2009, supra; in Tables 1 and 2, and in U.S. Pat. Application Publication No. US 2011/0262983 and PCT Publication No. WO 2011/123715.

In certain aspects, cellodextrin transporters of the disclosure include the polypeptides of: GenBank accession number CAZ81962.1 (Tuber melanosporum); GenBank accession number ABN65648.2 (Pichia stipitis); GenBank accession number EDR07962 (Laccaria bicolor); GenBank accession number BAE58341.1 (Aspergillus oryzae); GenBank accession number DAA06789.1 (Saccharomyces cerevisiae HXT1); GenBank accession number CAA30053.1 (Kluyveromyces lactis LACP) Joint Genome Institute (JGI) protein ID (PID) number PID 136620 (S1)(Phanerochaete chrysosporium); Joint Genome Institute (JGI) protein ID (PID) number PID 115604 (JGI) (S2) (Postia placenta); NCBI Reference Sequence XP_{—}001268541.1 (Aspergillus clavatus); NCBI Reference Sequence XP_{—}001387231 (LAC2, Pichia stipitis). The P. chrysosporium and P. placenta genomes can be accessed at http://genome.jgipsf.org/Phchr1/Phchr1.home.html and http://genome.jgipsf.org/Pospl1/Pospl1.home.html, respectively.

In certain aspects, the cellodextrin transporter of the host cell has at least about 20%, at least about 30%, at least about 40%, at least about 50%, at least about 55%, at least about 60%, at least about 65%, at least about 70%, at least about 75%, at least about 80%, at least about 85%, at least about 90%, at least about 92%, at least about 94%, at least about 96%, at least about 98%, at least about 99%, or 100% amino acid residue sequence identity to the polypeptide corresponding to GenBank accession number CAZ81962.1 (Tuber melanosporum), GenBank accession number ABN65648.2 (Pichia stipitis), GenBank accession number EDR07962 (Laccaria bicolor), GenBank accession number BAE58341.1 (Aspergillus oryzae), GenBank accession number DAA06789.1 (Saccharomyces cerevisiae HXT1), GenBank accession number CAA30053.1 (Kluyveromyces lactis LACP), Joint Genome Institute (JGI) protein ID (PID) number PID 136620 (S1)(Phanerochaete chrysosporium), Joint Genome Institute (JGI) protein ID (PID) number PID 115604 (JGI) (S2) (Postia placenta), NCBI Reference Sequence XP_{—}001268541.1 (Aspergillus clavatus), or NCBI Reference Sequence XP_{—}001387231 (LAC2, Pichia stipitis).

Cellodextrin Transporter Motifs

In one aspect, a cellodextrin transporter is a member of Major Facilitator Superfamily (MFS) sugar transporters. Members of the MFS (Transporter Classification #2.A.1) of transporters almost always consist of 12 transmembrane α-helices, with an intracellular N- and C-terminus (S. S. Pao, et al., Microbiol Mol Biol Rev 62, 1 (March, 1998)). While the primary sequence of MFS transporters varies widely, all are thought to share the tertiary structure of the E. coli lactose permease (LacY) (J. Abramson et al., Science 301, 610 (Aug. 1, 2003)), and the E. coli Pi/glycerol-3-phospate (GlpT) (Y. Huang, et al., Science 301, 616 (Aug. 1, 2003)). In these examples the six N- and C-terminal helices form two distinct domains connected by a long cytoplasmic loop between helices 6 and 7. This symmetry corresponds to a duplication event thought to have given rise to the MFS. Substrate binds within a hydrophilic cavity formed by helices 1, 2, 4, and 5 of the N-terminal domain, and helices 7, 8, 10, and 11 of the C-terminal domain. This cavity is stabilized by helices 3, 6, 9, and 12.

The Sugar Transporter family of the MFS (Transporter Classification #2.A.1.1) is defined by motifs found in transmembrane helices 6 and 12 (PESPR (SEQ ID NO: 9)/PETK (SEQ ID NO: 10)), and loops 2 and 8 (GRR/GRK) (M. C. Maiden, et al., Nature 325, 641 (Feb. 12-18, 1987)). The entire Hidden Markov Model (HMM) for this family can be viewed at pfam.janelia.org/family/PF00083#tabview=tab3. PROSITE (N. Hulo et al., Nucleic Acids Res 34, D227 (Jan. 1, 2006)) uses two motifs to identify members of this family. The first is [LIVMSTAG]-[LIVMFSAG]-{SH}-{RDE}-[LIVMSA]-[DE]-{TD}-[LIVMFYWA]-G-R-[RK]-x(4,6)-[GSTA] (SEQ ID NO: 11). The second is [LIVMF]-x-G-[LIVMFA]-{V}-x-G-{KP}-x(7)-[LIFY]-x(2)-[EQ]-x(6)-[RK] (SEQ ID NO: 12). As an example of how to read a PROSITE motif, the following motif, [AC]-x-V-x(4)-{ED}, is translated as: [Ala or Cys]-any-Val-any-any-any-any-{any but Glu or Asp} (SEQ ID NO: 13).

Multiple sequence alignments produced in T-COFFEE between putative cellodextrin transporter orthologs and confirmed cellodextrin transporters identified conserved motifs. The conserved motifs were defined using PROSITE notation. Transmembrane helix 1 contains the motif, [LIVM]-Y-[FL]-x(13)-[YF]-D (SEQ ID NO: 1). Transmembrane helix 2 contains the motif, [YF]-x(2)-G-x(5)-[PVF]-x(6)-[DQ] (SEQ ID NO: 2). The loop connecting transmembrane helix 2 and transmembrane helix 3 contains the motif, G-R-[RK] (SEQ ID NO: 3). Transmembrane helix 5 contains the motif, R-x(6)-[YF]-N (SEQ ID NO: 4). Transmembrane helix 6 contains the motif, WR-[IVLA]-P-x(3)-Q (SEQ ID NO: 5). The sequence between transmembrane helix 6 and transmembrane helix 7 contains the motif, P-E-S-P-R-x-L-x(8)-A-x(3)-L-x(2)-Y-H (SEQ ID NO: 6). Transmembrane helix 7 contains the motif, F-[GST]-Q-x-S-G-N-x-[LIV] (SEQ ID NO: 7). Transmembrane helix 10 and transmembrane helix 11 and the sequence between them contains the motif, L-x(3)-[YIV]-x(2)-E-x-L-x(4)-R-[GA]-K-G (SEQ ID NO: 8).

Accordingly, certain aspects of the present disclosure relate to recombinant cellodextrin transporters, or functional fragments thereof, that contain one or more of the disclosed conserved motifs. In certain embodiments, the recombinant cellodextrin transporter, or functional fragment thereof, includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 1 contains SEQ ID NO: 1. In other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 2 contains SEQ ID NO: 2. In still other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and a loop connecting transmembrane α-helix 2 and transmembrane α-helix 3 contains SEQ ID NO: 3. In yet other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 5 contains SEQ ID NO: 4. In other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 6 contains SEQ ID NO: 5. In still other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and sequence between transmembrane α-helix 6 and transmembrane α-helix 7 contains SEQ ID NO: 6. In yet other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 7 contains SEQ ID NO: 7. In other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 10 and transmembrane α-helix 11 and the sequence between them contain SEQ ID NO: 8.

Moreover, each of the above described embodiments may be combined in any number of combinations. A recombinant cellodextrin transporter according to any of the above embodiments may include a polypeptide containing 1, 2, 3, 4, 5, 6, or 7 of any of SEQ ID NOs: 1-8, or the polypeptide may contain all of SEQ ID NOs: 1-8. For example, a recombinant cellodextrin transporter may include a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, where transmembrane α-helix 1 contains SEQ ID NO: 1, a loop connecting transmembrane α-helix 2 and transmembrane α-helix 3 contains SEQ ID NO: 3, and transmembrane α-helix 7 contains SEQ ID NO: 7. Or, in another example, a recombinant cellodextrin transporter may include a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, where transmembrane α-helix 2 contains SEQ ID NO: 2, transmembrane α-helix 3 contains SEQ ID NO: 3, transmembrane α-helix 6 contains SEQ ID NO: 5, and transmembrane α-helix 10 and transmembrane α-helix 11 and the sequence between them contain SEQ ID NO: 8.

Mutant Cellodextrin Transporters

Other aspects of the present disclosure relate to mutant cellodextrin transporters that may be used to increase the function and/or activity of a cellodextrin transporter of the present disclosure. Mutant cellodextrin transporters may be produced by mutating a polynucleotide encoding a cellodextrin transporter of the present disclosure. In some embodiments, a mutant cellodextrin transporter of the present disclosure may contain at least one mutation that includes, without limitation, a point mutation, a missense mutation, a substitution mutation, a frameshift mutation, an insertion mutation, a duplication mutation, an amplification mutation, a translocation mutation, or an inversion mutation that results in a cellodextrin transporter with increased function and/or activity.

Methods of generating at least one mutation in a cellodextrin transporter of interest are well known in the art and include, without limitation, random mutagenesis and screening, site-directed mutagenesis, PCR mutagenesis, insertional mutagenesis, chemical mutagenesis, and irradiation.

In some embodiments, the mutant cellodextrin transporter contains one or more amino acid substitutions. For example, a cellodextrin transporter of the present disclosure may contain an amino acid substitution at one or more positions corresponding to positions of the amino acid sequence of Cdt-1 (SEQ ID NO: 33). Suitable one or more positions include, without limitation, a position corresponding to amino acid 91 of SEQ ID NO: 33, a position corresponding to amino acid 104 of SEQ ID NO: 33, a position corresponding to amino acid 170 of SEQ ID NO: 33, a position corresponding to amino acid 174 of SEQ ID NO: 33, a position corresponding to amino acid 194 of SEQ ID NO: 33, a position corresponding to amino acid 213 of SEQ ID NO: 33, a position corresponding to amino acid 335 of SEQ ID NO: 33, and combinations thereof.

In one non-limiting example, the amino acid substitution at one or more positions are a glycine (G) to alanine (A) substitution at a position corresponding to amino acid 91 of SEQ ID NO: 33; a glutamine (Q) to alanine (A) substitution at a position corresponding to amino acid 104 of SEQ ID NO: 33; a phenylalanine (F) to alanine (A) substitution at a position corresponding to amino acid 170 of SEQ ID NO: 33; am arginine (R) to alanine (A) substitution at a position corresponding to amino acid 174 of SEQ ID NO: 33; a glutamate (E) to alanine (A) substitution at a position corresponding to amino acid 194 of SEQ ID NO: 33; a phenylalanie (F) to lysine (L) substitution at a position corresponding to amino acid 213 of SEQ ID NO: 33; a phenylalanie (F) to alanine (A) substitution at a position corresponding to amino acid 335 of SEQ ID NO: 33; or combinations thereof. In certain preferred embodiments, the amino acid substitution at one or more positions is a glycine (G) to alanine (A) substitution at a position corresponding to amino acid 91 of SEQ ID NO: 33 and/or a phenylalanie (F) to lysine (L) substitution at a position corresponding to amino acid 213 of SEQ ID NO: 33.

In some embodiments, the increased function and/or activity of a mutant cellodextrin transporter results in a host cell that consumes cellodextrin at a rate faster than the rate of cellodextrin consumption in a cell lacking the mutant cellodextrin transporter. For example, the rate of cellodextrin consumption in a host cell containing a mutant cellodextrin transporter may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 225%, at least 250%, at least 275%, at least 300%, or at least a higher percentage faster than the rate of cellodextrin consumption in a host cell containing a corresponding wild-type cellodextrin transporter.

β-Glucosidase

In certain embodiments, the host cells further contain a recombinant nucleotide where the nucleotide encodes a polypeptide containing at least a catalytic domain of a β-glucosidase. As used herein, β-glucosidase refers to a β-D-glucoside glucohydrolase (E.C. 3.2.1.21), which catalyzes the hydrolysis of terminal non-reducing β-D-glucose residues with the release of β-D-glucose. A catalytic domain of β-glucosidase has β-glucosidase activity as determined, for example, according to the basic procedure described by Venturi et al., J. Basic Microb. 42 (1) 55-66, 2002. A catalytic domain of a β-glucosidase is any domain that catalyzes the hydrolysis of terminal non-reducing residues in β-D-glucosides with release of glucose. In some aspects, the β-glucosidase is a glycosyl hydrolase family 1 member. Members of this group can be identified by the motif, [LIVMFSTC]-[LIVFYS]-[LIV]-[LIVMST]-E-N-G-[LIVMFAR]-[CSAGN] (SEQ ID NO: 14). Here, E is the catalytic glutamate (webpage expasy.org/cgi-bin/prosite-search-ac?PD0000495). In certain aspects, the polynucleotide encoding a catalytic domain of β-glucosidase is heterologous to the host cell. In some aspects, the catalytic domain of β-glucosidase is located intracellularly in the host cell. In some aspects, the β-glucosidase is from N. crassa, and in some aspects, the β-glucosidase is NCU00130 (GH1-1). In certain embodiments, the β-glucosidase may be an ortholog of NCU00130. Examples of orthologs of NCU00130 include, without limitation (listed with GenBank Accession numbers): T. melanosporum, CAZ82985.1; A. oryzae, BAE57671.1; P. placenta, EED81359.1; P. chrysosporium, BAE87009.1; Kluyveromyces lactis, CAG99696.1; Laccaria bicolor, EDR09330; Clavispora lusitaniae, EEQ37997.1; Aspergillus aculeatus, D64088 and Pichia stipitis, ABN67130.1. Other β-glucosidases could be used include those from the glycosyl hydrolase family 3. These β-glucosidases can be identified by the following motif according to PROSITE: [LIVM](2)-[KR]-x-[EQKRD]-x(4)-G-[LIVMFTC]-[LIVT]-[LIVMF]-[ST]-D-x(2)-[SGADNIT] (SEQ ID NO: 15). Here D is the catalytic aspartate. Typically, any β-glucosidase may be used that contains the conserved domain of β-glucosidase/6-phospho-β-glucosidase/β-galactosidase found in NCBI sequence COG2723. Catalytic domains from specific β-glucosidases may be preferred depending on the cellodextrin transporter contained in the host cell.

### Methods of Producing and Culturing Host Cells of the Disclosure

Methods of producing and culturing host cells of the disclosure may include the introduction or transfer of expression vectors containing the recombinant polynucleotides of the disclosure into the host cell. Such methods for transferring expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host cell. Also, microinjection of the nucleic acid sequences provides the ability to transfect host cells. Other means, such as lipid complexes, liposomes, and dendrimers, may also be employed. Those of ordinary skill in the art can transfect a host cell with a desired sequence using these or other methods.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host, is integrated into the genome and replicated together with the chromosome(s) into which it has been integrated. Furthermore, a single vector or plasmid or two or more vectors or plasmids which together contain the total DNA to be introduced into the genome of the host, or a transposon may be used.

The vectors preferably contain one or more selectable markers which permit easy selection of transformed hosts. A selectable marker is a gene the product of which provides, for example, biocide or viral resistance, resistance to heavy metals, prototrophy to auxotrophs, and the like. Selection of bacterial cells may be based upon antimicrobial resistance that has been conferred by genes such as the amp, gpt, neo, and hyg genes.

Suitable markers for S. cerevisiae hosts are, for example, ADE2, HIS3, LEU2, LYS2, MET3, TRP1, and URA3. Selectable markers for use in a filamentous fungal host include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in Aspergillus are the amdS and pyrG genes of Aspergillus nidulans or Aspergillus oryzae and the bar gene of Streptomyces hygroscopicus. Preferred for use in Trichoderma are bar and amdS.

The vectors preferably contain an element(s) that permits integration of the vector into the host's genome or autonomous replication of the vector in the cell independent of the genome.

For integration into the host genome, the vector may rely on the gene's sequence or any other element of the vector for integration of the vector into the genome by homologous or nonhomologous recombination. Alternatively, the vector may contain additional nucleotide sequences for directing integration by homologous recombination into the genome of the host. The additional nucleotide sequences enable the vector to be integrated into the host genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host. Furthermore, the integrational elements may be non-encoding or encoding nucleotide sequences. On the other hand, the vector may be integrated into the genome of the host by non-homologous recombination.

For autonomous replication, the vector may further comprise an origin of replication enabling the vector to replicate autonomously in the host in question. The origin of replication may be any plasmid replicator mediating autonomous replication which functions in a cell. The term “origin of replication” or “plasmid replicator” is defined herein as a sequence that enables a plasmid or vector to replicate in vivo. Examples of origins of replication for use in a yeast host are the 2 micron origin of replication, ARS1, ARS4, the combination of ARS1 and CEN3, and the combination of ARS4 and CEN6. Examples of origins of replication useful in a filamentous fungal cell are AMA1 and ANSI (WO 00/24883). Isolation of the AMA1 gene and construction of plasmids or vectors including the gene can be accomplished according to the methods disclosed in WO 00/24883.

For other hosts, transformation procedures may be found, for example, in J. D. Read, et al., Applied and Environmental Microbiology, August 2007, p. 5088-5096, for Kluyveromyces, in O. Delgado, et al., FEMS Microbiology Letters 132, 1995, 23-26, for Zymomonas, in U.S. Pat. No. 7,501,275 for Pichia stipitis, and in WO 2008/040387 for Clostridium.

More than one copy of a gene may be inserted into the host to increase production of the gene product. An increase in the copy number of the gene can be obtained by integrating at least one additional copy of the gene into the host genome or by including an amplifiable selectable marker gene with the nucleotide sequence where cells containing amplified copies of the selectable marker gene, and thereby additional copies of the gene, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

The host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. Growth of a host cell in a medium may involve the process of fermentation. Methods of the disclosure may include culturing the host cell such that recombinant nucleic acids in the cell are expressed. For microbial hosts, this process entails culturing the cells in a suitable medium. In some aspects, cells are grown at 30° C. in appropriate media. Growth media of the present disclosure include, for example and without limitation, common commercially prepared media such as Luria Bertani (LB) broth, Sabouraud Dextrose (SD) broth or Yeast medium (YM) broth. Other defined or synthetic growth media may also be used and the appropriate medium for growth of the particular host cell will be known by someone skilled in the art of microbiology or fermentation science. Temperature ranges and other conditions suitable for growth are known in the art.

According to some aspects of the disclosure, the culture media contains a carbon source for the host cell. Such a “carbon source” generally refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, etc. These include, for example, various monosaccharides such as glucose, oligosaccharides, polysaccharides, a biomass polymer such as cellulose or hemicellulose, xylose, arabinose, disaccharides, such as sucrose, saturated or unsaturated fatty acids, succinate, lactate, acetate, ethanol, etc., or mixtures thereof. The carbon source can additionally be a product of photosynthesis, including, but not limited to glucose.

In some embodiments, the carbon source is a biomass polymer such as cellulose or hemicellulose. “A biomass polymer” as described herein is any polymer contained in biological material. The biological material may be living or dead. A biomass polymer includes, for example, cellulose, xylan, xylose, hemicellulose, lignin, mannan, and other materials commonly found in biomass. Non-limiting examples of sources of a biomass polymer include grasses (e.g., switchgrass, Miscanthus), rice hulls, bagasse, cotton, jute, hemp, flax, bamboo, sisal, abaca, straw, leaves, grass clippings, corn stover, corn cobs, distillers grains, legume plants, sorghum, sugar cane, sugar beet pulp, wood chips, sawdust, and biomass crops (e.g., Crambe).

In addition to an appropriate carbon source, media must contain suitable minerals, salts, cofactors, buffers and other components, known to those skilled in the art, suitable for the growth of the cultures and promotion of the enzymatic pathways necessary for the fermentation of various sugars and the production of hydrocarbons and hydrocarbon derivatives. Reactions may be performed under aerobic or anaerobic conditions where aerobic, anoxic, or anaerobic conditions are preferred based on the requirements of the microorganism. As the host cell grows and/or multiplies, expression of the enzymes, transporters, or other proteins necessary for growth on various sugars or biomass polymers, sugar fermentation, or synthesis of hydrocarbons or hydrocarbon derivatives is affected.

### Method of Fermenting β-D-Glucose-Containing Mixtures

In one embodiment, methods of fermenting β-D-glucose-containing mixtures are provided. In one aspect, the β-D-glucose is obtained through the chemical or enzymatic hydrolysis of cellulose or cellodextrins into β-D-glucose.

In one aspect, a method of fermenting β-D-glucose-containing mixture includes a first step wherein a β-D-glucose-containing mixture is contacted with one or more recombinant polypeptides disclosed herein having glucose mutarotase activity. In certain aspects, the polypeptide having D-glucose mutarotase activity has at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the GAL10/YBR019C, YHR210C, YNR071C, or NCU09705 polypeptide. The method includes a second step wherein after contacting the β-D-glucose-containing mixture with a polypeptide having D-glucose mutarotase activity, or concomitant with contacting the β-D-glucose-containing mixture with a polypeptide having D-glucose mutarotase activity, the β-D-glucose-containing mixture is contacted with a host cell. In one aspect, the host cell is S. cerevisiae. The host cell is cultured with the β-D-glucose-containing mixture under conditions that support fermentation, and wherein the host cell consumes more β-D-glucose of the β-D-glucose-containing mixture than are consumed by a cell that contacts β-D-glucose-containing mixture that has not been contacted with a recombinant polypeptide having glucose mutarotase activity. As would be understood by one having skill in the art, contacting the β-D-glucose-containing mixture with one or more polypeptides having glucose mutarotase activity may reduce the content of β-D-glucose in that mixture, by converting it to α-D-glucose.

In another aspect, a method of fermenting β-D-glucose-containing mixtures includes a first step wherein a β-D-glucose-containing mixture is contacted with a host cell, where the host cell contains a recombinant polynucleotide encoding a polypeptide disclosed herein having glucose mutarotase activity. In one aspect, the host cell is S. cerevisiae. In certain aspects, the recombinant polynucleotide encodes a polypeptide having at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the GAL10/YBR019C, YHR210C, YNR071C, or NCU09705 polypeptide. The method includes a second step of culturing the cell with the β-D-glucose-containing mixture such that the recombinant polynucleotide is expressed, and under conditions that support fermentation, and where the host cell consumes more β-D-glucose of the β-D-glucose-containing mixture than are consumed by a cell that does not contain the recombinant polynucleotide encoding a polypeptide having glucose mutarotase activity. Preferably, the host cell containing the recombinant polynucleotide encoding a polypeptide having glucose mutarotase activity and the host cell that does not contain the recombinant polynucleotide encoding a polypeptide having glucose mutarotase activity will otherwise be identical in genetic background.

### Method of Fermenting Cellodextrin-Containing Mixtures

In one embodiment, the present disclosure provides a method of fermenting cellodextrin-containing mixtures. In one aspect, cellodextrins are obtained through the chemical or enzymatic hydrolysis of cellulose. In one aspect, cellodextrins are cellobiose.

In one aspect, a method of fermenting cellodextrin-containing mixtures includes a first step of providing a host cell, where the host cell contains a recombinant polynucleotide encoding a polypeptide disclosed herein having glucose mutarotase activity. In one aspect, the host cell is S. cerevisiae. In certain aspects, the recombinant polynucleotide encodes a polypeptide having at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the GAL10/YBR019C, YHR210C, YNR071C, or NCU09705 polypeptide. The method includes a second step of culturing the cell with the cellodextrin-containing mixtures such that the recombinant polynucleotide is expressed, and under conditions that support fermentation.

In another aspect, a method of fermenting cellodextrin-containing mixtures includes a first step of providing a host cell, where the host cell contains a recombinant polynucleotide encoding a polypeptide having glucose mutarotase activity and a recombinant polynucleotide encoding one or more cellodextrin transporters. In one aspect, the host cell is S. cerevisiae. In certain aspects, a recombinant polynucleotide encodes a polypeptide having glucose mutarotase activity having at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the GAL10/YBR019C, YHR210C, YNR071C, or NCU09705 polypeptide. In certain aspects, a recombinant polynucleotide encodes a cellodextrin transporter having at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the NCU00801 or NCU08114 polypeptides. In one aspect, the host cell further contains a recombinant polynucleotide encoding a polypeptide containing at least a catalytic domain of a β-glucosidase. Such recombinant polynucleotides are useful for host cells lacking the endogenous ability to utilize cellodextrins. In one aspect, the catalytic domain of the β-glucosidase is intracellular. In one aspect, the β-glucosidase is from Neurospora crassa. In one aspect, the β-glucosidase is encoded by NCU00130.

The method includes a second step of culturing the cell with the cellodextrin-containing mixture such that the recombinant polynucleotides are expressed, and under conditions that support fermentation.

### Method of Increasing Consumption of Cellodextrins During Fermentation of Cellodextrin-Containing Mixtures

The present disclosure further provides a method of increasing the consumption of cellodextrins during fermentation of cellodextrin-containing mixtures. In one aspect, the cellodextrins are obtained through the chemical or enzymatic hydrolysis of cellulose. In one aspect, the cellodextrins are cellobiose.

In one aspect, a method of increasing the consumption of cellodextrins during fermentation of cellodextrin-containing mixtures includes a first step of providing a host cell, where the host cell contains a recombinant polynucleotide encoding a polypeptide disclosed herein having glucose mutarotase activity and a recombinant polynucleotide encoding one or more cellodextrin transporters. In one aspect, the host cell is S. cerevisiae. In certain aspects, a recombinant polynucleotide encodes a polypeptide having glucose mutarotase activity having at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% amino acid identity to the GAL10/YBR019C, YHR210C, YNR071C, or NCU09705 polypeptide. In certain aspects, a recombinant polynucleotide encodes a cellodextrin transporter having at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% amino acid identity to the NCU00801 or NCU08114 polypeptides. In one aspect, the host cell containing further contains a recombinant polynucleotide encoding a polypeptide containing at least a catalytic domain of a β-glucosidase. Such recombinant polynucleotides are useful for host cells lacking the endogenous ability to utilize cellodextrins. In one aspect, the catalytic domain of the β-glucosidase is intracellular. In one aspect, the β-glucosidase is from Neurospora crassa. In one aspect, the β-glucosidase is encoded by NCU00130.

The method includes a second step of culturing the cell with the cellodextrin-containing mixture such that the recombinant polynucleotides are expressed, and under conditions that support fermentation, and where the host cell consumes more cellodextrins of the cellodextrin-containing mixture than are consumed by a cell that does not contain the recombinant polynucleotide encoding a polypeptide having glucose mutarotase activity. Preferably, the host cell containing the recombinant polynucleotide encoding a polypeptide having glucose mutarotase activity and the host cell that does not contain the recombinant polynucleotide encoding a polypeptide having glucose mutarotase activity will otherwise be identical in genetic background.

The consumption of cellodextrins by a host cell may be measured by any method known to one of skill in the art. Typically, consumption of cellodextrins by a cell will be measured by evaluating concentration of cellodextrins in medium in which the cell was growing by high performance liquid chromatography (HPLC). Preferably, the host cell containing the recombinant polynucleotide encoding a polypeptide having glucose mutarotase activity and the host cell that does not contain the recombinant polynucleotide encoding a polypeptide having glucose mutarotase activity will otherwise be identical in genetic background. Media containing cellodextrins may have resulted from enzymatic treatment of biomass polymers such as cellulose.

### Method of Increasing Production of a Chemical During Fermentation of Cellodextrin-Containing Mixtures

The present disclosure further provides a method of increasing production of a chemical during fermentation of cellodextrin-containing mixtures. In one aspect, the cellodextrins are obtained through the chemical or enzymatic hydrolysis of cellulose. In one aspect, the cellodextrins are cellobiose.

In one aspect, a method of increasing production of a chemical during fermentation of cellodextrin-containing mixtures includes a first step of providing a host cell, where the host cell contains a recombinant polynucleotide encoding a polypeptide disclosed herein having glucose mutarotase activity and a recombinant polynucleotide encoding one or more cellodextrin transporters. In one aspect, the host cell is S. cerevisiae. In certain aspects, a recombinant polynucleotide encodes a polypeptide having glucose mutarotase activity having at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the GAL10/YBR019C, YHR210C, YNR071C, or NCU09705 polypeptide. In certain aspects, a recombinant polynucleotide encodes a cellodextrin transporter having at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% amino acid identity to the NCU00801 or NCU08114 polypeptides. In one aspect, the host cell further contains a recombinant polynucleotide encoding a polypeptide containing at least a catalytic domain of a β-glucosidase. Such recombinant polynucleotides are useful for host cells lacking the endogenous ability to utilize cellodextrins. In one aspect, the catalytic domain of the β-glucosidase is intracellular. In one aspect, the β-glucosidase is from Neurospora crassa. In one aspect, the β-glucosidase is encoded by NCU00130.

The method includes a second step of culturing the cell with the cellodextrin-containing mixture such that the recombinant polynucleotides are expressed, and under conditions that support fermentation, and where the host cell produces more of the chemical during the fermentation of the cellodextrin-containing mixture than is produced by a cell that does not contain the recombinant polynucleotide encoding a polypeptide having glucose mutarotase activity. Preferably, the host cell containing the recombinant polynucleotide encoding a polypeptide having glucose mutarotase activity and the host cell that does not contain the recombinant polynucleotide encoding a polypeptide having glucose mutarotase activity will otherwise be identical in genetic background.

In one aspect, chemicals that may be produced during fermentation of cellodextrin-containing mixtures includes any product that may be made by a microbial host during fermentation of sugar. In one aspect, a chemical that may be produced during fermentation of cellodextrin-containing mixtures is an alcohol. In one aspect, a chemical that may be produced during fermentation of cellodextrin-containing mixtures is ethanol, propanol, or butanol.

The production of chemicals by a host cell may be measured by any method known to one of skill in the art. Typically, production of a chemical such as an alcohol by a cell will be measured by evaluating the concentration of the chemical in medium in which the cell was growing, by high performance liquid chromatography (HPLC).

### Method of Increasing Cell Growth Rate During Fermentation of Cellodextrin-Containing Mixtures

The present disclosure further provides a method of increasing cell growth rate during fermentation of cellodextrin-containing mixtures. In one aspect, cellodextrins are obtained through the chemical or enzymatic hydrolysis of cellulose. In one aspect, the cellodextrins are cellobiose.

In one aspect, a method of increasing cell growth rate during fermentation of cellodextrin-containing mixtures includes a first step of providing a host cell, where the host cell contains a recombinant polynucleotide encoding a polypeptide disclosed herein having glucose mutarotase activity and a recombinant polynucleotide encoding one or more cellodextrin transporters. In one aspect, the host cell is S. cerevisiae. In certain aspects, a recombinant polynucleotide encodes a polypeptide having glucose mutarotase activity having at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% amino acid identity to the GAL10/YBR019C, YHR210C, YNR071C, or NCU09705 polypeptide. In certain aspects, a recombinant polynucleotide encodes a cellodextrin transporter having at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% amino acid identity to the NCU00801 or NCU08114 polypeptides. In one aspect, the host cell further contains a recombinant polynucleotide encoding a polypeptide containing at least a catalytic domain of a β-glucosidase. Such recombinant polynucleotides are useful for host cells lacking the endogenous ability to utilize cellodextrins. In one aspect, the catalytic domain of the β-glucosidase is intracellular. In one aspect, the β-glucosidase is from Neurospora crassa. In one aspect, the β-glucosidase is encoded by NCU00130.

The method includes a second step of culturing the cell with the cellodextrin-containing mixture such that the recombinant polynucleotides are expressed, and under conditions that support fermentation, and where the host cell has an increased growth rate during the fermentation of the cellodextrin-containing mixture as compared to the growth rate of a cell that does not contain the recombinant polynucleotide encoding a polypeptide having glucose mutarotase activity. Preferably, the host cell containing the recombinant polynucleotide encoding a polypeptide having glucose mutarotase activity and the host cell that does not contain the recombinant polynucleotide encoding a polypeptide having glucose mutarotase activity will otherwise be identical in genetic background.

The growth rate of a host cell may be measured by any method known to one of skill in the art. Typically, growth rate of a cell will be measured by evaluating cell concentration in suspension by optical density.

Methods of Consolidated Bioprocessing

Further provided herein are methods for converting cellulose-containing materials to a fermentation production, by consolidated bioprocessing. Consolidated bioprocessing combines enzyme generation, biomass hydrolysis, and biofuel production into a single stage. In one aspect, a method for converting a cellulose-containing material into a fermentation product by consolidated bioprocessing includes the steps of: A) contacting a cellulose-containing material with a cell having recombinant nucleic acids encoding one or more cellulases, one or more cellodextrin transporters, one or more β-glucosidases, and one or more polypeptides having glucose mutarotase activity disclosed herein; and, B) incubating the cellulose-containing material with the cell expressing the recombinant nucleic acids under conditions that support cellulose degradation and fermentation, in order to produce a fermentation product.

Fermentation products that may be produced from sugars obtained from the degradation of cellulose-containing materials include, without limitation, ethanol, n-propanol, n-butanol, iso-butanol, 3-methyl-1-butanol, 2-methyl-1-butanol, 3-methyl-1-pentanol, and octanol.

### Methods of Generating Cellodextrin and β-D-Glucose

In one aspect, cellodextrins and β-D-glucose of the present disclosure are generated from cellulose. Cellulose is composed of long chains of β(1-4) linked D-glucose molecules. The hydrolysis of cellulose into smaller molecules may result in the production of molecules of approximately two to six β-D-glucose molecules linked together (“cellodextrins”), or of individual β-D-glucose molecules. In one aspect, cellodextrins generated by the hydrolysis of cellulose are cellobiose.

In one aspect, cellulose is obtained from plant biomass. In one aspect, cellulose is obtained from algae. In one aspect, cellulose is obtained from fungi. In another aspect, cellulose is obtained from bacterial biofilms.

In one aspect, cellulose is obtained from lignocellulose. Major components of lignocellulose are cellulose, hemicellulose, and lignin. Methods for making cellulose available from lignocellulose are known to those of skill in the art, and include physical and chemical processes. In one aspect, cellulose is made available from lignocellulose through acid hydrolysis. In one aspect, cellulose is made available from lignocellulose through steam explosion. In one aspect, cellulose is made available from lignocellulose through ammonia fiber expansion (AFEX).

Cellulose may be hydrolyzed into cellodextrins and β-D-glucose molecules by enzymatic or chemical means.

Cellulose may be chemically hydrolyzed into cellodextrins and β-D-glucose molecules by treating the cellulose with an acid. In one aspect, cellulose is treated with acid at atmospheric pressure and room temperature. In one aspect, cellulose is treated with acid at greater than atmospheric pressure and a temperature greater than 40° C.

Cellulose may be enzymatically hydrolyzed into cellodextrins and β-D-glucose molecules by treating the cellulose with cellulases. Cellulases may include, for example, endoglucanases, exoglucanases, and beta-glucosidases. In one aspect, cellulases are recombinant cellulases. In one aspect, cellulases are thermostable cellulases. In one aspect, contains a carbohydrate binding domain (CBD) at the C-terminal end of the protein.

In some aspects, cellulases may be isolated directly from an organism that naturally expresses cellulases. In some aspects, cellulases may be produced recombinantly, using a cellulase gene from an organism that naturally expresses cellulases in an expression vector in a host cell. Types of organisms that express cellulases include, for example, fungi and bacteria.

Fungi that express cellulases include, without limitation, Acremonium cellulolyticus, Aspergillus acculeatus, Aspergillus fumigatus, Aspergillus niger, Fusarium solani, Hypocrea jecornia (Trichoderma reesei), Irpex lacteus, Penicillium funmiculosum, Phanerochaete chrysosporium, Schizophyllum commune, Sclerotium rolfsii, Sporotrichum cellulophilum, Talaromyces emersonii, Thielavia terrestris, Trichoderma koningii, and Trichoderma viride.

Bacteria that express cellulases include, without limitation, Acidothermus cellulolyticus, Agrobacterium sp., Bacillus subtilis, Clostridium cellulovorans, Clostridium thermocellum, Paenibacillus polymyxa, Pectobacterium chrysanthami, Pyrococcus furiousus, Ruminococcus albus, Streptomyces sp., Thermoactinomyces sp., Thermobifida fusca, Thermomonospora curvata, Thermotoga maritime, and Thermotoga neapolitana.

It is to be understood that, while the disclosure has been described in conjunction with the specific embodiments thereof, the foregoing description is intended to illustrate and not limit the scope of the invention. Other aspects, advantages, and modifications within the scope of the disclosure will be apparent to those skilled in the art to which the disclosure pertains.

### EXAMPLES

The following Examples are merely illustrative and are not meant to limit any aspects of the present disclosure in any way.

### Example 1

### Overexpression of Genes Encoding Aldose 1-Epimerases (AEP) in Cellobiose Fermenting S. cerevisiae Strain D452-BT

To investigate the effect of AEP overexpression on cellobiose consumption, various genes coding for AEP were cloned and over-expressed in an engineered S. cerevisiae expressing a cellodextrin transporter (cdt-1) and an intracellular β-glucosidase (gh1-1). Five genes coding for AEP were cloned from different microorganisms and introduced into a multi-copy plasmid (pRS423) under the control of a strong promoter (either the PGK promoter or the TEF promoter) (Table 1).

TABLE 1
Various AEP genes from S. cerevisiae, Pichia stipitis, and Escherichia coli.

| Construct name | Gene name | Source | Number |
|---|---|---|---|
| pRS423-GAL10-Sc | GAL10 | S. cerevisiae | YBR019C |
| pRS423-GAL10-YHR 210C | Putative epimerase | S. cerevisiae | YHR210C |
| pRS423-GAL10-YNR 071C | Putative epimerase | S. cerevisiae | YNR071C |
| pRS423-GAL10-Ps | GAL10 | P. stipitis | |
| pRS423-galM-Ec | galM | E. coli | |

Each construct was introduced into an engineered cellobiose utilizing S. cerevisiae D452-BT strain in which a cellodextrin transporter (cdt-1) and β-glucosidase (gh1-1) were over-expressed using multi-copy plasmids pRS426 and pRS425, respectively.

To compare the rates of cellobiose utilization and ethanol production, after pre-culture in minimum medium with cellobiose, those transformants were inoculated into 50 mL of YP-cellobiose (80 g/L) medium in 250 mL flasks with initial ODs of around 1.0. Fermentation experiments were performed at 100 rpm and 30° C. Cell growth was monitored by measuring optical density (OD) at 600 nm using UV-visible spectrophotometer (Biomate 5, Thermo, NY). Cellobiose, glucose, glycerol, acetate and ethanol concentrations were determined by high performance liquid chromatography (HPLC, Agilent Technologies 1200 Series) equipped with a refractive index detector. The Rezex ROA-Organic Acid H⁺ (8%) column (Phenomenex Inc., Torrance, Calif.) was used and the column was eluted with 0.005 N of H₂SO₄ as a mobile phase at a flow rate of 0.6 ml/min at 50° C.

### Comparison of Various Aldose-1-Epimerases on Cellobiose Utilization

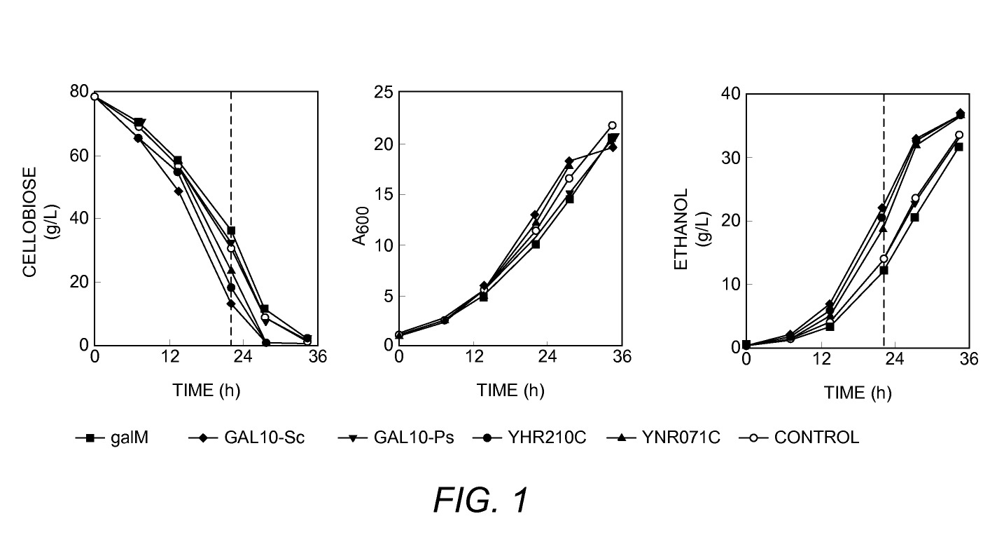

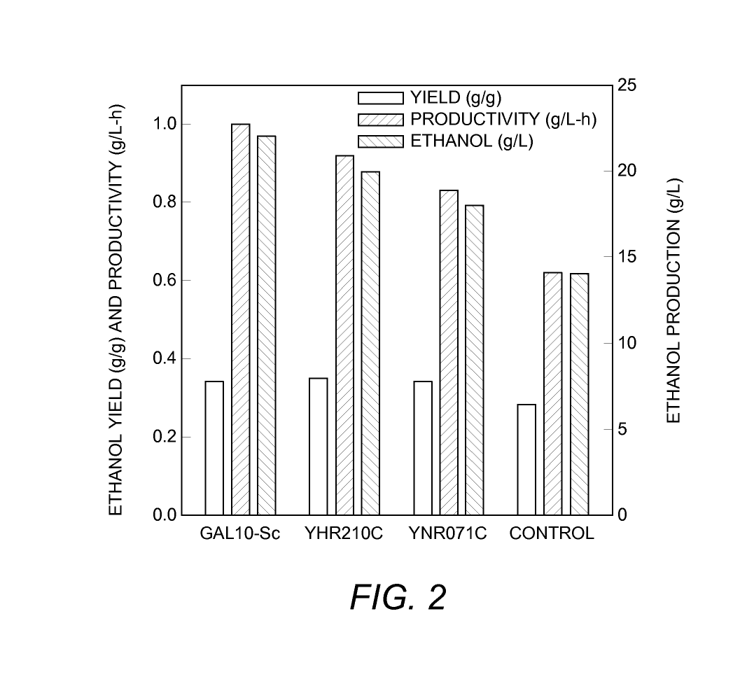

From the fermentation experiments using six newly constructed strains and the parental strain, we found that overexpression of GAL10 (YBR019C) and two putative alodose-1-epimerases coded by YHR210C and YNR071C from S. cerevisiae showed improved cellobiose consumption rates and ethanol production rates (FIG. 1). GAL10 from P. stipitis and galM from E. coli did not show any effects on cellobiose utilization (FIG. 1). At the time of 22 hour, the amounts of consumed cellobiose (65, 60, and 55 g/L) by the transformants with GAL10 (YBR019C), YHR210C, and YNR071C were much higher than that of control strain containing an empty plasmid (pRS423) (48 g/L) (Table 2). As a result, ethanol production also increased from 14 g/L to 22, 20, and 18 g/L by overexpression of GAL10 (YBR019C), YHR210C, and YNR071C, respectively. Higher cellobiose consumption rate and ethanol production rate by the overexpression of aldose-1-epimerase genes resulted in significant improvements in ethanol productivity (from 0.62 to 1.00, 0.92, and 0.83 g/L-hr).

TABLE 2
Comparison of cellobiose consumption and ethanol production
by AEP overexpression strains in 22 h fermentation.

| | | Consumed | | | |
|---|---|---|---|---|---|
| | OD | Cellobiose | Ethanol | Y_{EtOH} | P_{EtOH} |
| | (A₆₀₀) | (g/L) | (g/L) | (g/g) | (g/L-hr) |
| galM-Ec | 10 | 42 | 12 | 0.28 | 0.54 |
| GAL10-Sc | 13 | 65 | 22 | 0.34 | 1.00 |
| GAL10-Ps | 11 | 47 | 13 | 0.29 | 0.61 |
| YHR210C-Sc | 12 | 60 | 20 | 0.35 | 0.92 |
| YNR071C-Sc | 12. | 55 | 18 | 0.34 | 0.83 |
| Control-423 | 11 | 48 | 14 | 0.28 | 0.62 |

### Effect of GAL10 (YBR019C) Overexpression on Cellobiose Fermentation

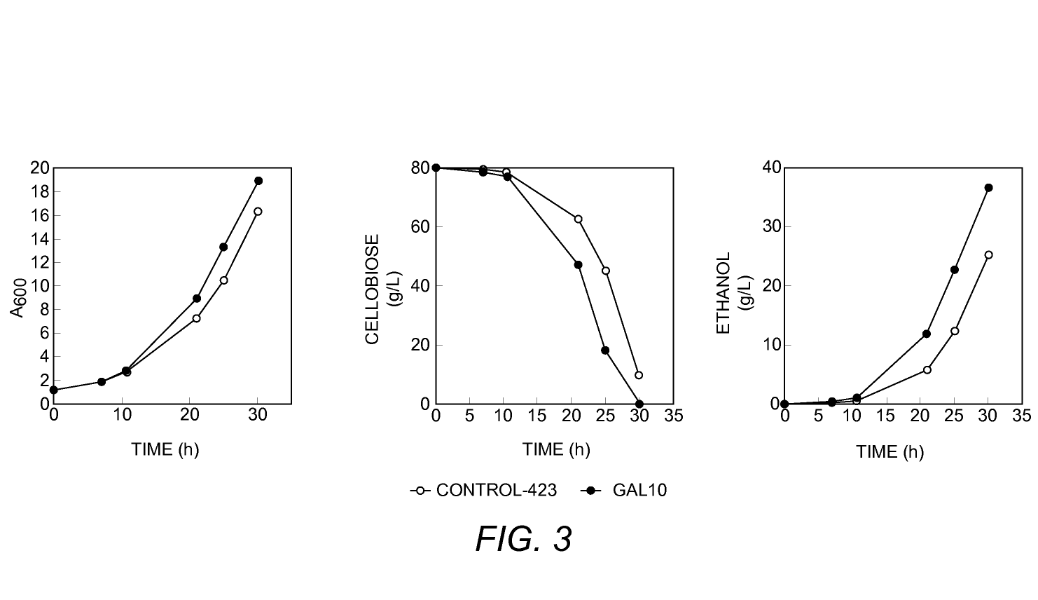

To confirm the effect of GAL10 (YBR019C) overexpression on cellobiose fermentation, we independently repeated fermentation experiments with GAL10 (YBR019C) overexpressed strain (D452-BT-423GAL10) and the parental strain (D452BT). Fermentations were performed in YP-cellobiose (80 g/L) medium and an oxygen limited condition at 30° C. As expected, D452-BT-423GAL10 showed higher cell growth, cellobiose consumption rate, and ethanol production rate. D452-BT-423GAL10 grew more (OD 19 vs. OD 16), consumed cellobiose more (80 g/L vs. 70 g/L), and produced ethanol more (37 g/L vs 25 g/L) than the control strain at 30 h (FIG. 3). As a result, ethanol productivity increased 50% (1.23 vs. 0.83 g/L·h) by the overexpression of GAL10 (YBR019C).

### Example 2

### Overexpression of Genes Encoding Aldose 1-Epimerases (AEP) in Cellobiose Fermenting S. cerevisiae Strain SL01

Two AEP genes were also cloned in multi-copy plasmid pRS424 and expressed in S. cerevisiae strain SL01, which contains a cellodextrin transporter (cdt-1) and β-glucosidase (gh1-1) in multi-copy plasmid pRS425 (Table 3). Each AEP gene was cloned with the HXT7 promoter and the HXT7 terminator. Empty plasmid pRS424 was also transformed into the SL01 strain as a negative control.

TABLE 3
Source of AEP genes overexpressed in the SL01 strain

| Construct name | Source | GenBank ID |
|---|---|---|
| scAEP | S. cerevisiae | YHR210C |
| ncAEP | N. crassa | NCU09705 |

Yeast strains were cultivated in synthetic dropout media to maintain plasmids (0.17% Difco yeast nitrogen base without amino acids and ammonium sulfate, 0.5% ammonium sulfate, 0.05% amino acid dropout mix). YPA medium (1% yeast extract, 2% peptone, 0.01% adenine hemisulfate) with 2% D-glucose was used to grow yeast strains. S. cerevisiae strains were grown in un-baffled shake-flasks at 30° C. and 100 rpm for fermentation. For each yeast strain, a single colony was first grown up in 2 mL SC-Ura-Leu medium plus 20 g/L glucose, and then inoculated into 50 mL of the same medium in a 250 mL shake flask to obtain enough cells for mixed sugar fermentation studies. After one day of growth, cells were spun down and inoculated into 50 mL of YPA medium supplemented with 80 g/L cellobiose. Sugars and ethanol concentrations were determined using Shimadzu HPLC equipped with a Bio-Rad HPX-87H column (Bio-Rad Laboratories, Hercules, Calif.) and Shimadzu RID-10A refractive index detector following the manufacturer's protocol. The HPX-87H column was kept at 65° C. using a Shimadzu CTO-20AC column oven. 0.5 mM sulfuric acid solution was used as mobile phase at a constant flow rate of 0.6 mL/min. 10 μL of filtered sample was injected into the HPLC system with a Shimadzu SIL-20AC HT auto sampler, and each run was stopped at 25 minutes after the injection. The concentrations of the sugars and ethanol were determined using a standard curve generated using a series of external standards. Each data point represented the mean of duplicate samples.

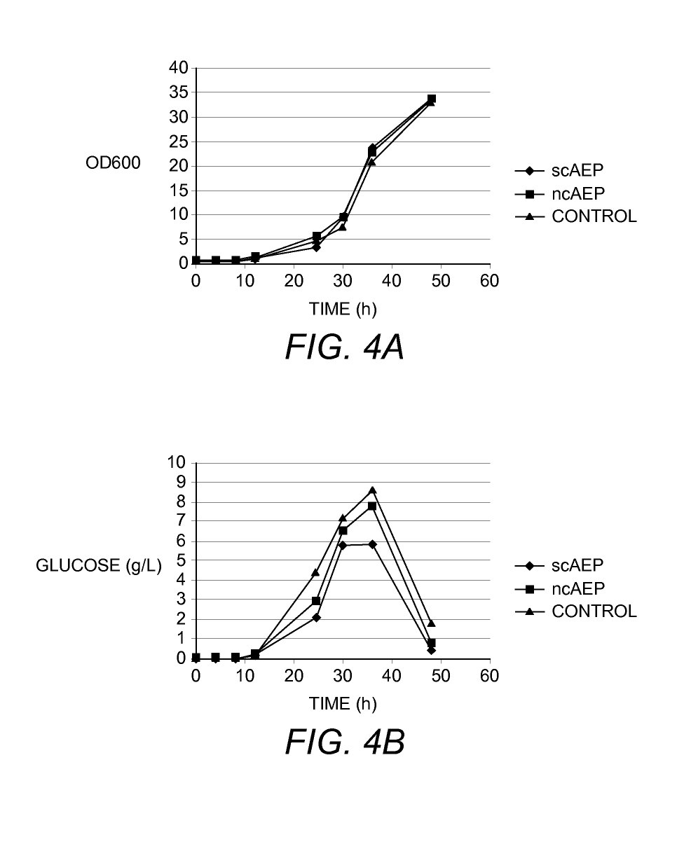

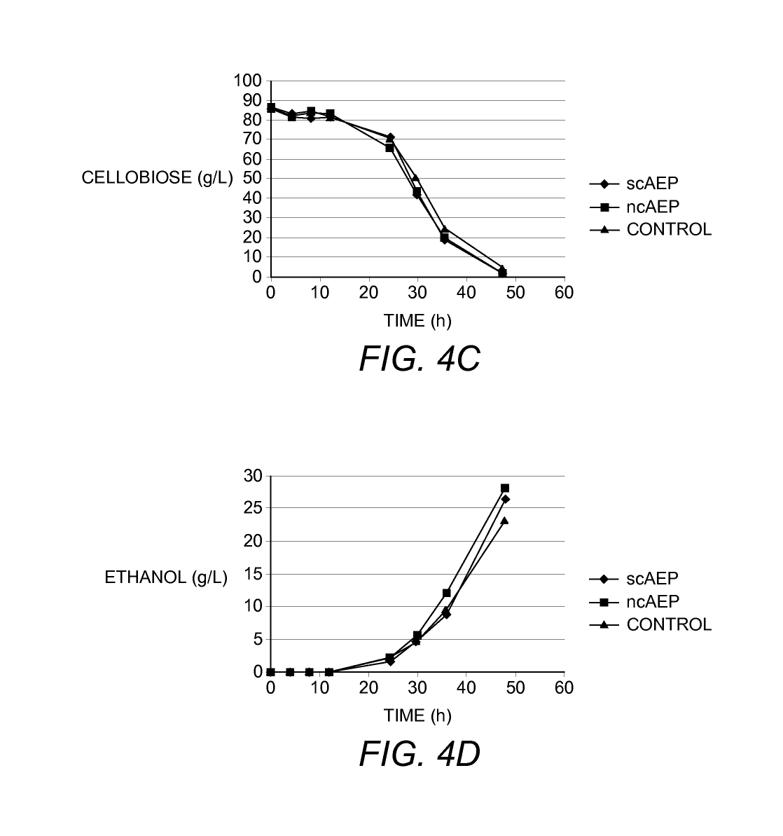

As shown in FIG. 4, overexpressed AEP genes showed slightly improved biomass growth, cellobiose consumption rate, ethanol production, and decreased glucose accumulation. The cellobiose consumption rate showed 9% and 8% increase in the scAEP-overexpressing strain and the ncAEP-overexpressing strain, respectively. The ethanol production rate was enhanced by 8% and 25% in the scAEP-overexpressing strain and the ncAEP-overexpressing strain, respectively. The glucose accumulation rate was decreased in both the scAEP-overexpressing strain and the ncAEP-overexpressing strain: at 48 h, the scAEP-overexpressing strain showed a 32% less glucose accumulation while the ncAEP-overexpressing strain showed a 9.5% less glucose accumulation than control strain (5.8, 7.8 and 8.6 g/L).

### Example 3

### S. cerevisiae Having One or Two AEP Genes Knocked Out

To further investigate the function of the aldose 1-epimerase genes in S. cerevisiae, a gene knockout strategy was used. Based on bioinformatics studies, three putative aldose-1-epimerase genes were identified in S. cerevisiae, including YHR210c, YNR071c, and YBR019c. To explore the different knockout combinations, we constructed three single AEP knockout strains, including ΔYHR210c, ΔYNR071c and ΔGAL10 (YBR019c), and two double AEP knockout strains including Δ(YHR210c+YNR071c) and Δ(YHR210c+GAL10). The AEP knockout strains were constructed in S. cerevisiae strain SL01, and all knockout strains contain the plasmid pRS425-(gh1-1)-cdt1, which encodes a cellodextrin transporter (cdt-1) and β-glucosidase (gh1-1).

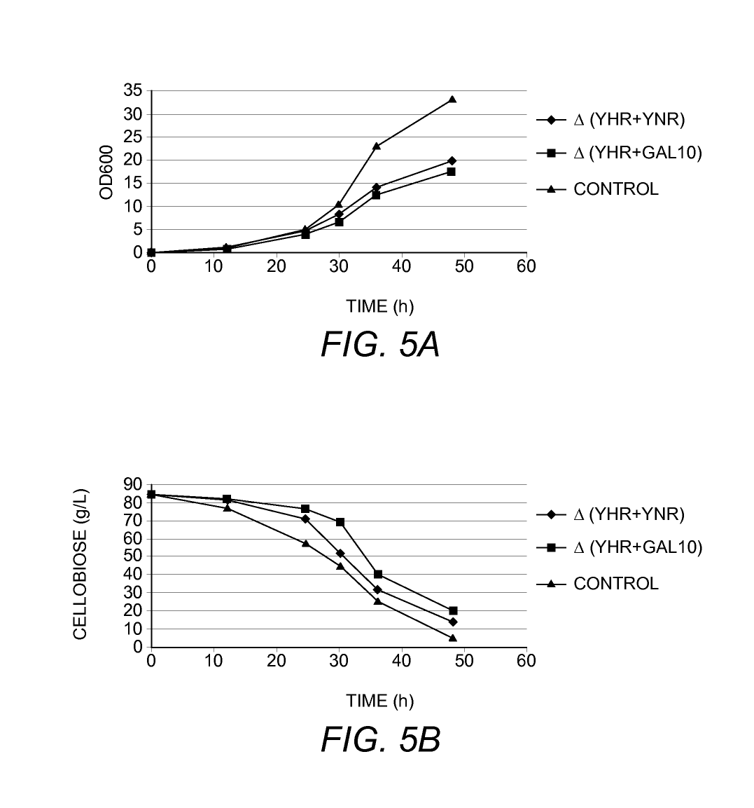

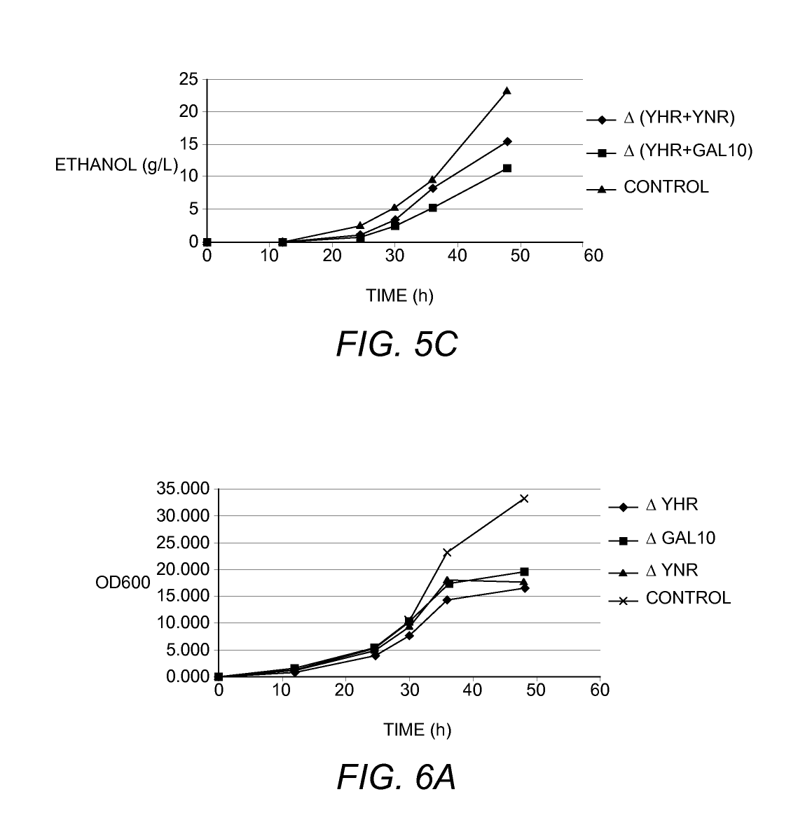

In cellobiose utilization, the two double knockout strains showed obvious reduction in fermentation capability (Table 4 and FIG. 5): at 48 h, the biomass production was significantly decreased from OD33 to OD19.7 (Δ(YHR210c+YNR071c)) or OD17.5 (Δ(YHR210c+GAL10)); the cellobiose consumption rate was decreased by 10% in Δ(YHR210c+YNR071c) strain, from 1.65 g/L/h to 1.48 g/L/h, and decreased by 18% in Δ(YHR210c+GAL10) strain from 1.65 g/L/h to 1.35 g/L/h; the ethanol production rate was decreased by 34% in Δ(YHR210c+YNR071c) strain, from 0.483 g/L/h to 0.318 g/L/h, and decreased by 52% in Δ(YHR210c+GAL10) strain from 0.483 g/L/h to 0.234 g/L/h.

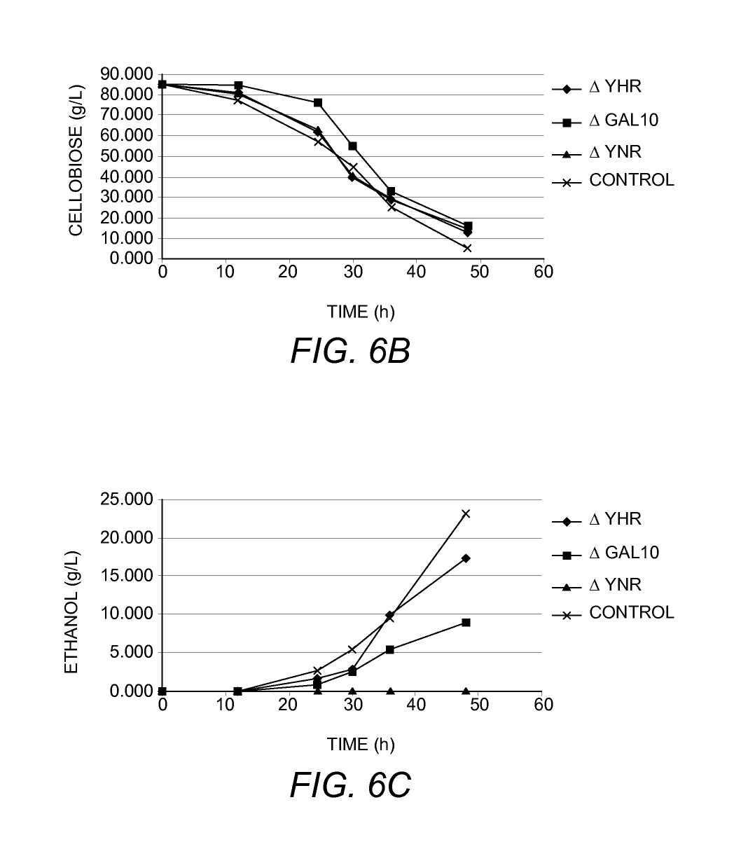

The three single knockout strains also showed some drawbacks in fermentation capability (FIG. 6): at 48 h, the biomass production was significantly decreased from OD33 to OD17 (ΔYHR210c), OD 19 (ΔGAL10) or OD 18 (ΔYNR071c); the cellobiose consumption rate was decreased from 1.65 g/L/h to 1.48 g/L/h (ΔYHR210c), 1.43 g/L/h (ΔGAL10) or 1.46 g/L/h (ΔYNR071c); the ethanol production rate was decreased from 0.483 g/L/h to 0.360 g/L/h (ΔYHR210c) or 0.185 g/L/h (ΔGAL10). In ΔYNR071c strain all ethanol was consumed as carbon source.

TABLE 4
Fermentation comparison in AEP-knockout strains

| | | Cellobiose | | Ethanol | |
|---|---|---|---|---|---|
| | | consumption | Ethanol | yield | P_{EtOH} |
| | OD | (g/L) | (g/L) | (g/g) | (g/L/h) |
| Δ(YHR210c + | 19.695 | 71.010 | 15.279 | 0.218 | 0.318 |
| YNR071c) | | | | | |
| Δ(YHR210c + | 17.498 | 64.903 | 11.223 | 0.179 | 0.234 |
| GAL10) | | | | | |
| Control | 33.078 | 79.223 | 23.161 | 0.300 | 0.483 |

A 24-hour lag time existed in the AEP knockout strains, especially in cellobiose consumption. During this period, little cellobiose was consumed by either of the double knockout strains, which means in vivo glucose production was repressed because of low expression level of aldose 1-epimerase, resulting in low cellobiose consumption and biomass production. The impact due to lack of aldose 1-epimerase existed during the total cultivation process, which results in low biomass production, ethanol production and cellobiose consumption at the end of cultivation. Since in the Δ(YHR210c+YNR071c) double knockout strain, the only functional aldose 1-epimerase gene is GAL10, and in Δ(YHR210c+GAL10) double knockout strain, the only functional aldose 1-epimerase gene is YNR071c, we can conclude that the expression level and catalytic ability of YNR071c is lower than GAL10, as Δ(YHR210c+GAL10) showed a slower growth rate and a slower cellobiose consumption rate than Δ(YHR210c+YNR071c).

### Example 4

### Comparison of Glucose and Cellobiose Fermentation Rates in S. cerevisiae Strain D452-BT Containing cdt-1 and gh1-1 Genes

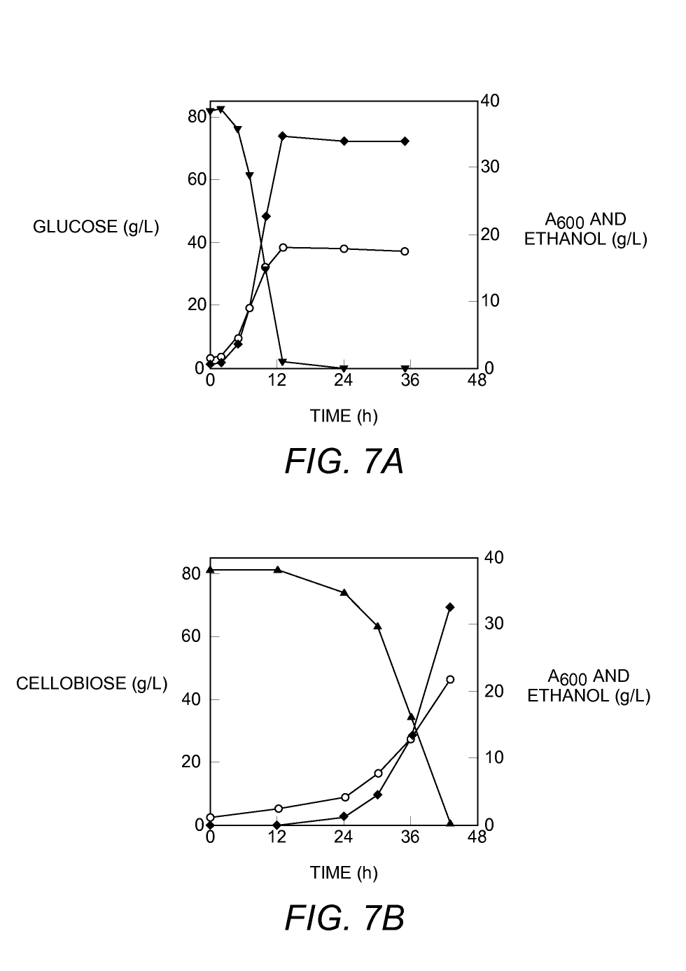

Introduction of both cellobiose transporter (cdt-1) and intracellular β-glucosidase (gh1-1) into S. cerevisiae enabled fermentation of cellobiose as a carbon source (Ha et al. 2011, supra; Li et al. 2010, supra). Because transported cellobiose is hydrolyzed into two molecules of glucose intracellularly, we can speculate that the cellobiose fermentation rate by the engineered S. cerevisiae might be similar with the glucose fermentation rate as long as cellobiose transport is not rate-limiting. However, cellobiose fermentation rates by engineered S. cerevisiae containing cdt-1 and gh1-1 were much lower than glucose fermentation rates. The engineered S. cerevisiae D452-BT consumed 80 g/L of cellobiose and produced 32.7 g/L of ethanol within 43 h, resulting in 0.41 g/g of ethanol yield and 1.9 g/L-h of cellobiose consumption rate (FIG. 7). However, the engineered S. cerevisiae D452-BT consumed 80 g/L of glucose and produced 34.7 g/L of ethanol within 13 h, resulted in 0.44 g/g of ethanol yield and 6.1 g/L-h of glucose consumption rate. Although final ethanol concentrations and ethanol yields were similar regardless of sugars (mono-glucose or di-glucose), the cellobiose consumption rate was three-fold lower than that of glucose. This result suggests that there might be unknown limiting steps for efficient cellobiose fermentation.

### Example 5

### Transcriptomic Analysis of Aldose 1-Epimerase Expression Level of Neurospora crassa Grown in Different Media

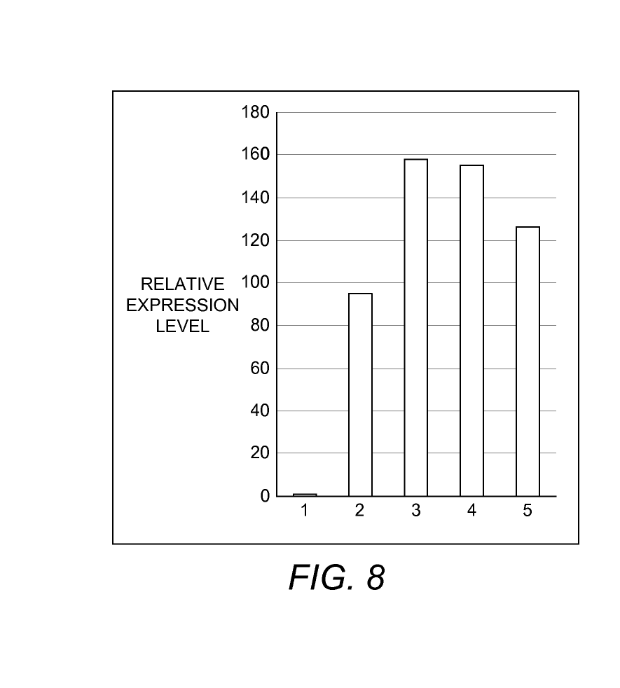

Because cellobiose transporter (cdt-1) and intracellular β-glucosidase (gh1-1) were cloned from cellulolytic fungi, Neurospora crassa, it may be possible to find gene targets to improve cellobiose fermentation by an engineered S. cerevisiae through exploring gene expression data obtained during cellulosic biomass utilization by N. crassa. According to previous transcript profiling study with N. crassa using both cellulosic substrates and monomeric sugars, the expression level of aldose 1-epimerase (NCU08398, AEP-Nc) was highly increased when N. crassa grew on Miscanthus hydrolyzate as compared to sucrose containing medium (Tian et al. 2009) (FIG. 8). This result suggested that higher expression of AEP-Nc was required for efficient intracellular cellobiose utilization through cellobiose transporter (cdt-1) and intracellular β-glucosidase (gh1-1). As such, we hypothesized that higher expression of AEP might improve cellobiose fermentation by engineered S. cerevisiae.

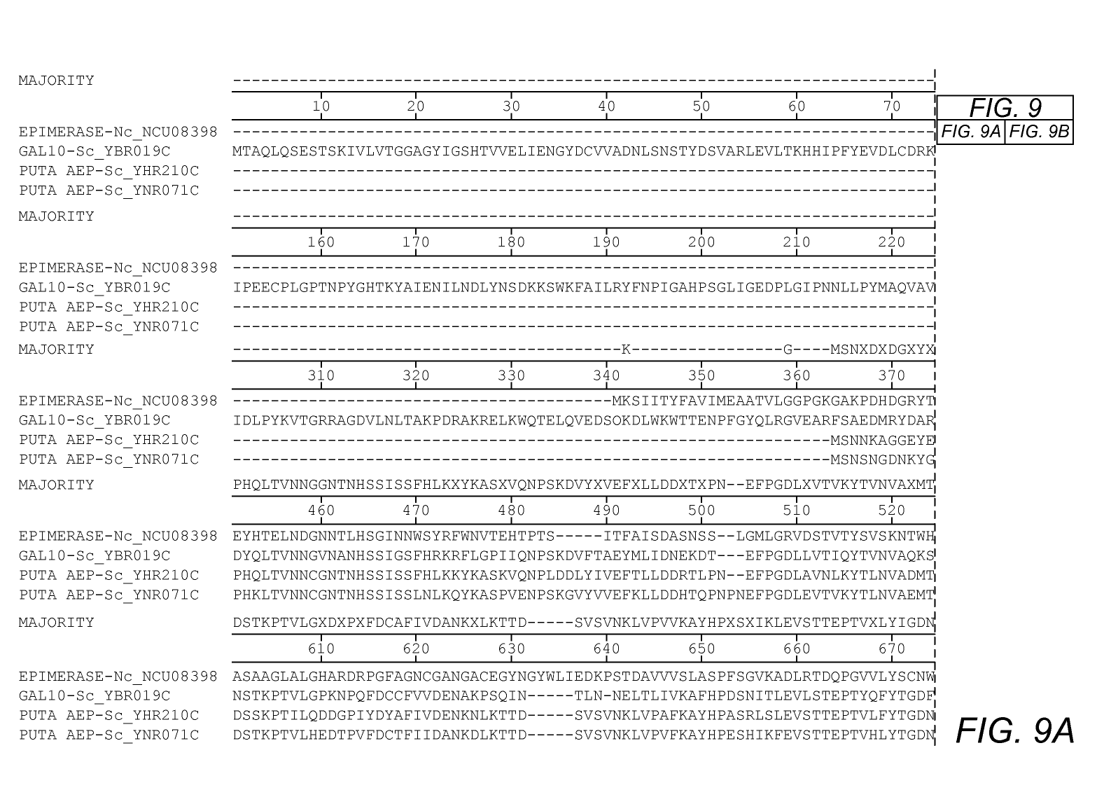

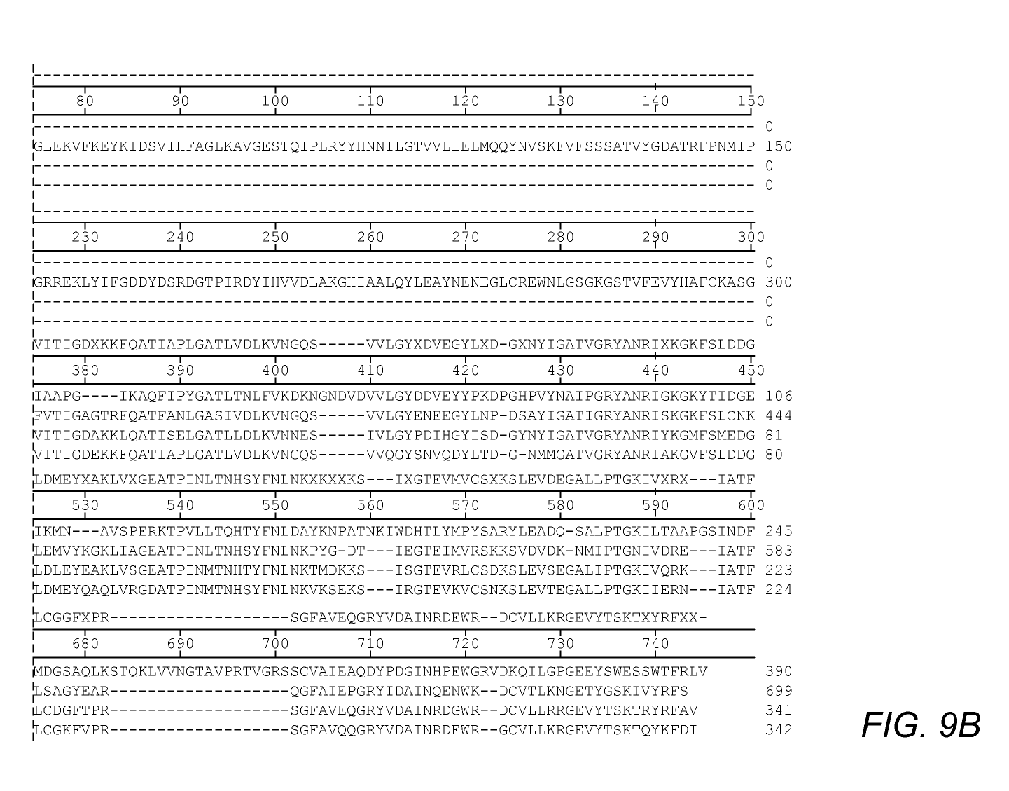

The GAL10 gene in S. cerevisiae codes for a bifunctional enzyme with AEP and UDP galactose 4-epimerase activities. Three dimensional structure analysis revealed that Ga110 possesses both a galactose 4-epimerase domain (N-terminal region) and an aldose 1-epimerase domain (C-terminal region) (Sharma and Malakar 2010). When aligned with AEP-Nc, Gal10 showed 24.7% sequence identify with the amino acid sequence of AEP-Nc (FIG. 9). Additionally, we found two putative AEP genes (YHR210C and YNR071C) which have high amino acid identities with Gal10 (50.6% and 51.0%, respectively). The amino acid sequences of YHR210C and YNR071C also have 24.2% and 26.6% sequence identity with that of AEP-Nc.

### Example 6

### Deletion of AEP Genes in S. cerevisiae Strain BY4741

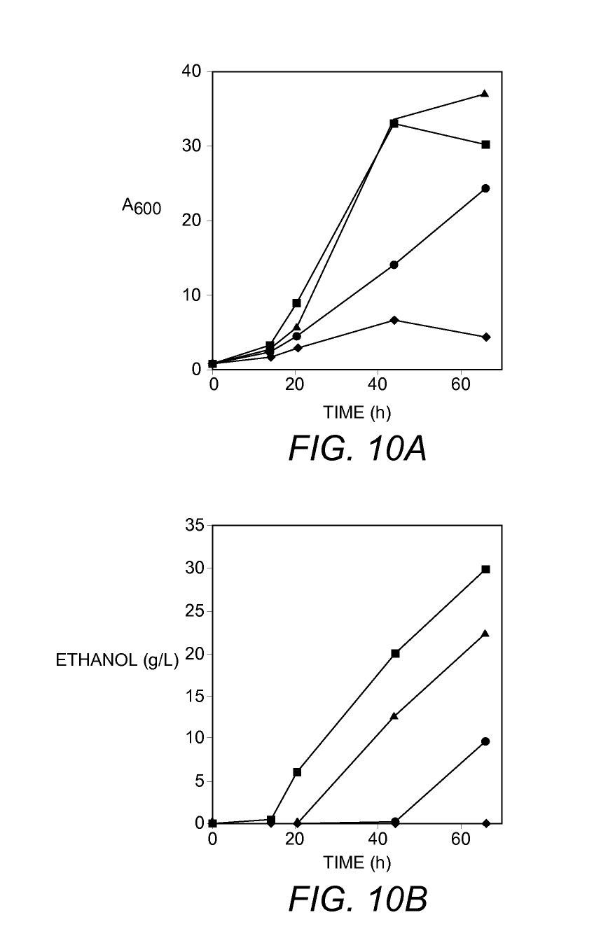

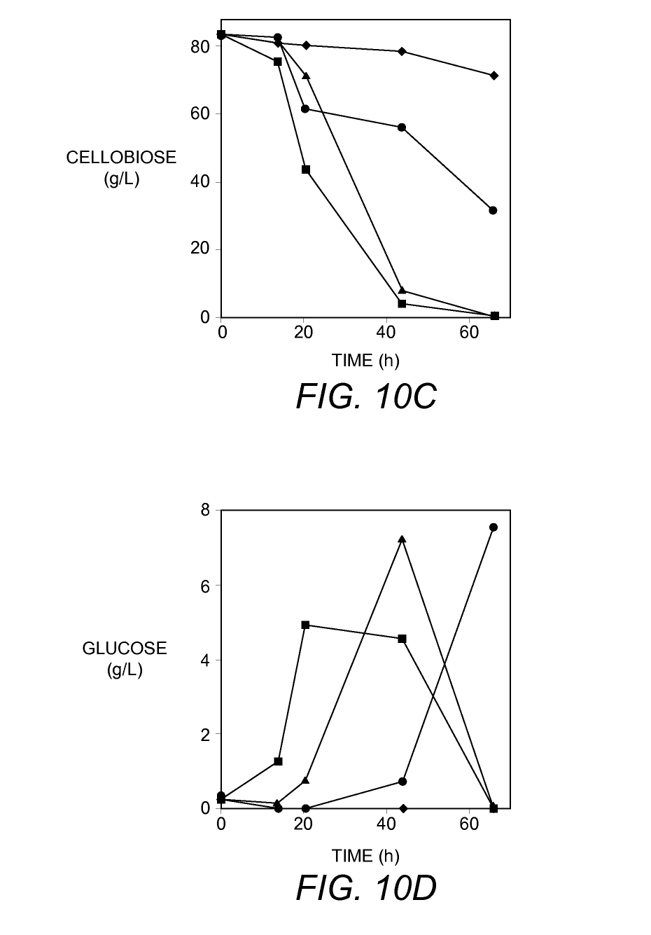

In order to further examine the function of putative AEPs in cellobiose utilization process, a plasmid harboring the cellobiose utilizing pathway was introduced into three BY 4741 knock-out strains which had YHR210C, YNR071C, or GAL10 gene deleted. When the resultant ΔYHR, ΔYNR, ΔGAL, and control strains with the cellobiose utilization pathway were grown, the ΔYHR and ΔYNR strains showed better cellobiose consumption than the wild type strain. Interestingly, the ΔGAL strain showed complete loss of growth on cellobiose (FIG. 10). The cellobiose consumption rates were enhanced from 0.78 g/L-h to either 1.26 g/L-h, or 1.25 g/L-h, in ΔYHR strain and ΔYNR strain, which represented either 60.3% or 59.9% improvement than the wild type strain respectively. As to glucose accumulation, the ΔYHR and ΔYNR strains showed higher glucose accumulation than the wild type strain, which was in proportional to the cellobiose consumption rate by the ΔYHR and ΔYNR strains. Thus, the accumulation of glucose is due to cellobiose consumption rather than the deletion of YHR210C or YNR071C genes. Considering the higher cellobiose consumption rate by the ΔYHR and ΔYNR strains, ethanol production was correlated to cellobiose consumption. Therefore, the ΔYHR strain showed the highest ethanol productivity among all strains. However, in the ΔGAL strain, little cellobiose was consumed. At the end of fermentation, only 12.0 g/L cellobiose consumption was observed. As a result, no glucose accumulation or ethanol production was observed by the ΔGAL strain.

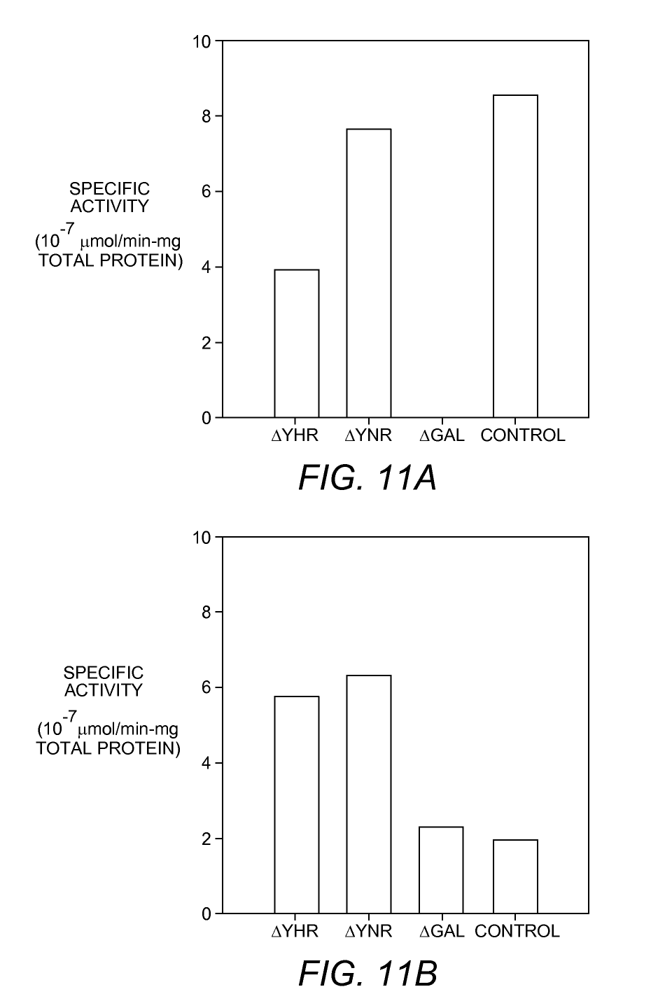

To further probe the function of AEPs, we determined the AEP enzymatic activity of the AEP deletion strains (FIG. 11). When those AEP deleted strains were grown on cellobiose as a sole carbon source, the specific mutarotase activity of the ΔYHR strain was 45.7% of that of the control strain, while the specific mutarotase activity of the ΔYNR strain was 89.5% of that of the control strain. No AEP activity was detected in the ΔGAL strain (FIG. 11A). AEP activities tested were quite different using glucose as a sole carbon source. The specific mutarotase activities of ΔYHR strain and ΔYNR strain were almost on the same level, which was approximate 3-fold of the activities of ΔGAL strain and control strain (FIG. 11B).

### Example 7

### S. cerevisiae Strains Having AEP Gene Overexpression

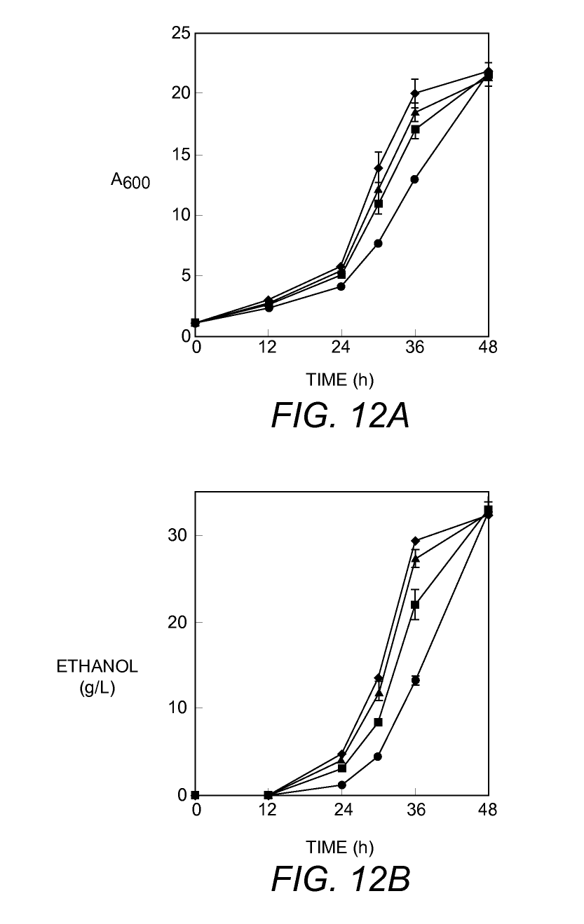

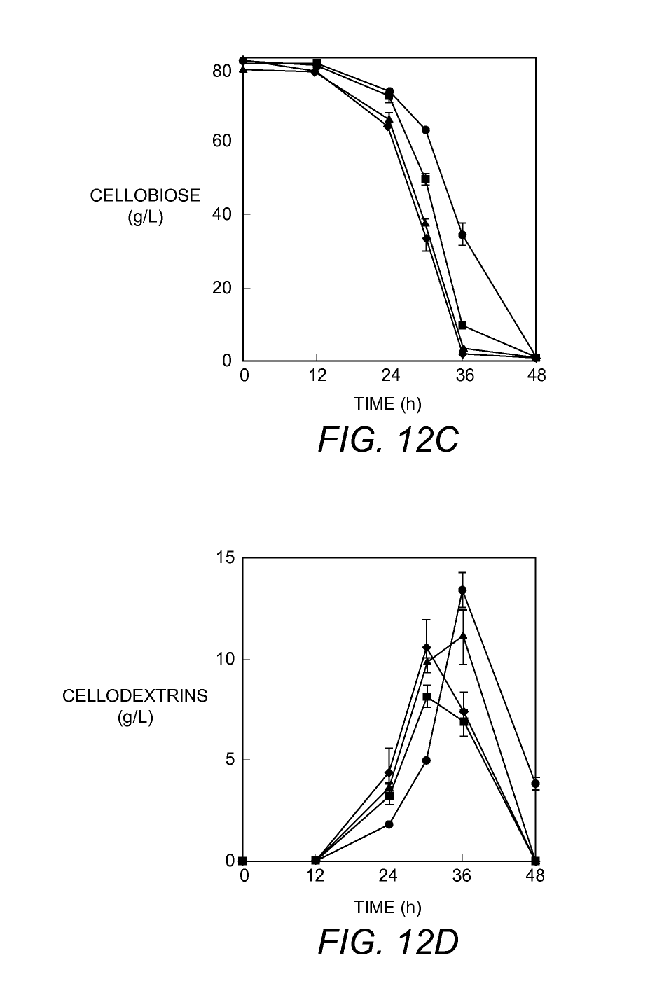

In order to determine the effect of AEP overexpression, we introduced overexpression cassettes facilitating strong expression levels of AEP genes (GAL10, YHR210C, and YNR071C) into two engineered S. cerevisiae strains with different strain backgrounds. For AEP overexpression, a pRS423 vector having the AEP under control of the TEF1 promoter and CYC1 terminator was used. For the cellobiose utilizing pathway, the constructs pRS426-cdt-1 and pRS425-gh1-1, having genes under control of the PGK promoter and CYC1 terminator were used (Galazka et al. 2010, supra; Ha et al. 2011, supra). While there was no improvement in cellobiose fermentation when S. cerevisiae BY4741 was used as a parental strain (data not shown), improved cellobiose consumption rate and ethanol production rate were observed when S. cerevisiae D452-BT was used as a parental strain (FIG. 12). While all overexpressions of GAL10, YHR210C, and YNR071 led to improved cellobiose fermentation, the GAL10 overexpressing strain showed highest cellobiose fermentation rates and ethanol production rates as compared to the YHR210C and YNR071 overexpressing strains. Additionally, cellodextrin accumulations decreased after introduction of AEP genes. As a result, the GAL10 overexpressing strain showed a 72% higher cellobiose consumption rate and a 119% ethanol production rate as compared to a control strain at 36 hour.

Discussion of Examples

For cellulosic biofuels production, efficient utilization of the two most abundant sugars in cellulosic biomass, glucose and xylose, is desirable. In the past two decades, there have been a lot of efforts to create an engineered Saccharomyces cerevisiae for efficient glucose and xylose utilization through genetic and metabolic engineering (Tantirungkij et al., J. Ferment. Bioeng. 75 (2):83-88, 1993; Kotter and Ciriacy, Appl. Microbiol. Biotechnol. 38 (6):776-783, 1993; Ho et al., Appl. Environ. Microbiol. 64 (5):1852-1859, 1998; Jin et al., Appl. Environ. Microbiol. 69 (1):495-503, 2003; Hahn-Hagerdal et al., Adv. Biochem. Eng./Biotechnol. 108: 147-177, 2007). However, the sequential utilization of glucose and xylose has several limitations such as low ethanol productivity and low ethanol yield from xylose (Jeffries and Jin, Appl. Microbiol. Biotechnol. 63 (5):495-509, 2004; Hahn-Hagerdal et al. 2007, supra). To address these issues, we developed a new strategy for co-fermentation of cellobiose and xylose, which drastically improved ethanol production yield and productivity (Ha et al. 2011, supra; Li et al. 2010, supra). However, the cellobiose fermentation rate is three fold slower than that of glucose albeit they have similar ethanol yields. This result suggested that there might be unknown limiting steps for efficient cellobiose utilization. Glucose induction is known to be initiated by signaling mechanisms from cell membranes (Rolland et al., FEMS Yeast Res. 2 (2):183-201, 2002,; Santangelo, Microbiol. Mol. Biol. R. 70 (1):253-282, 2006; Gancedo, Microbiol. Mol. Biol. R. 62 (2):334-361, 1998). However, in case of cellobiose fermentation, since glucose is produced inside of the cell from cellobiose by intracellular β-glucosidase, the normal glucose signaling mechanisms may not be efficient, resulting in slow cellobiose utilization.

Neurospora crassa is known to utilize cellobiose and both the cellobiose transporter (CDT-1) and the intracellular β-glucosidase (GH1-1) from N. crassa have been cloned and characterized (Galazka et al., Science 330 (6000):84-86, 2010). Therefore, we examined the transcriptomic analysis data from N. crassa to figure out the limiting steps in cellobiose utilization by an engineered S. cerevisiae. Interestingly, we found that the expression level of AEP was 160 times higher in the minimum medium containing Miscanthus hydrolyzate compared to that in the sucrose containing medium. This result suggested that the high expression level of AEP may facilitate cellobiose utilization by N. crassa. In the engineered cellobiose utilization, β form glucose is produced by β-glucosidase, however, the interconversion of β form sugar to a form sugar may be not high enough in vivo even though the interconversion of the sugar anomers occurs spontaneously in vitro (Fekete et al., Proc. Natl. Acad. Sci. U.S.A. 105 (20):7141-7146, 2008; Bouffard et al., J. Mol. Biol. 244 (3):269-278, 1994). Because yeast is known to prefer α-glucose, the activity of AEP could be rate-limiting for efficient cellobiose fermentation.

Using the AEP gene from N. crassa (AEP-Nc) as a probe sequence for BLAST search, we identified the GAL10 gene in S. cerevisiae that encodes a bifunctional enzyme with AEP and UDP galactose 4-epimerase activities. In additional, we identified two more putative AEP genes (YHR210C and YNR071C) in S. cerevisiae. GAL10, YHR210C, and YNR071C have 24.7%, 24.2%, and 26.6% amino acid sequence identity with AEP-Nc, respectively. The YHR210C gene was annotated as a putative protein of unknown function. However, its sequence similarity to GAL10 was reported (Majumdar et al., Eur. J. Biochem. 271 (4):753-759, 2004). For the YNR071C gene, its function is unknown. YHR210C and YNR071C also have 50.6% and 51.0% amino acid sequence identity with GAL10, respectively.

Based on our studies on the AEP knock-out strains, it was concluded that the GAL10 epimerase plays a dominant role in cellobiose utilization. In BY4741 strain, with GAL10 gene knocked out, there was hardly any growth on cellobiose and no AEP activity was observed at all. However, the results from the enzymatic assays suggest that although GAL10 is the dominant AEP, its expression is repressed by the expression of the other two AEPs. Under cellobiose fermentation condition where a high level of AEP is required, GAL10 is expressed efficiently and the GAL10 deletion led to limited AEP enzyme activity and no cell growth. Under glucose fermentation condition, since the GAL10 expression is repressed by glucose, the ΔGAL strain and the control strain showed almost identical enzymatic activities. Deleted YHR210C and YNR071C cannot repress the expression of GAL10, which explains the high AEP activities observed in ΔYHR and ΔYNR strains. Based on this analysis, a complex AEP regulation system may exist in BY4741 strain: when glucose interconversion is needed, the expression of the dominant GAL10 is activated; and when glucose is sufficient, low activity AEPs such as YHR210C and YNR071C are expressed but the GAL10 expression is repressed.

Further investigation of AEP overexpression was limited by the poor fermentation performance of BY4741 strains. Therefore, a faster growth strain, D452-BT was used. By introducing an AEP gene into S. cerevisiae D452-BT, cellobiose fermentation rates were significantly improved. While the control strain with empty pRS423 vector consumed only 47 g/L of cellobiose in 36 h, the strain overexpressing one of the three AEP genes consumed 72-80 g/L in 36 h. The difference of ethanol production between the control strain and the strain overexpressing one of the three AEP genes is even more significant. Ethanol production by strains overexpressing GAL10, YHR210C, and YNR071C are 123%, 110%, and 69% higher than that of the control strain for 36 h, respectively. Additionally, the maximum cellodextrin accumulations were decreased from 13 g/L to 7-11 g/L by overexpression of an AEP gene. Taken together, by introduction of an AEP gene into the engineered S. cerevisiae D452-BT with a cellobiose fermentation pathway, not only the cell growth rate, cellobiose consumption rate, and ethanol production rate were improved but also cellodextrin accumulations were decreased.

Materials and Methods for the Examples

Materials and methods for the above Examples include:

Strains and Plasmid Constructions

S. cerevisiae D452-BT (MATα, leu2, his3, ura3, can1) and S. cerevisiae BY4741 (MATα, his3, leu2, met15, ura3) were used for engineering of cellobiose metabolism. Three AEP knock-out strains were purchased from Open Biosystems (Huntsville, Ala.). Escherichia coli DH5 (F⁻ recA1 endA1 hsdR17 [rK⁻ mK⁺] supE44 thi-1 gyrA relA1) (Invitrogen, Gaithersburg, Md.) was used for gene cloning and manipulation. For expressions of β-glucosidase (gh1-1) and cellodextrin transporter (cdt-1) from Neurospora crassa, two open reading frames (cdt-1 and gh1-1) were placed between the PGK promoter and the CYC1 terminator to generate pRS426-CDT-1 and pRS425-BGL, respectively (Galazka et al. 2010, supra; Ha et al. 2011, supra). One AEP gene (GAL10) and two putative AEP genes (YHR210C and YNR071C) were placed between the TEF 1 promoter and the CYC1 terminator to generate pRS423-AEP.

The pRS425 plasmid (New England Biolabs, Ipwich, Mass.) was used to construct the plasmid harboring gh1-1 and cdt-1 used in the AEP knock-out strains. The PYK1 promoter and the ADH1 terminator were added to the N-terminus and C-terminus of the cellobiose transporter, respectively, while the TEF1 promoter and the PGK1 terminator were added to the N-terminus and C-terminus of the β-glucosidase, respectively.

Medium and Culture Conditions

E. coli was grown in Luria-Bertani medium; 50 μg/mL of ampicillin was added to the medium when required. Yeast strains were routinely cultivated at 30° C. in YP medium (10 g/L yeast extract, 20 g/L Bacto peptone) with 20 g/L of glucose. To select transformants using an amino acid auxotrophic marker, yeast synthetic complete (YSC) medium was used, which contained 6.7 g/L of yeast nitrogen base plus 20 g/L of glucose, 20 g/L of agar, and CSM-Leu-Trp-Ura that supply appropriate nucleotides and amino acids (Bio 101, Vista, Calif.).

Fermentation Experiments

Yeast cells were grown in YSC medium containing 20 g/L of glucose to prepare inoculums for cellobiose fermentation. Cells at the mid-exponential phase were harvested and inoculated after washing twice by sterilized water. All of the flask fermentation experiments were performed using 50 mL of YP medium containing 80 g/L of cellobiose in 250 mL flask at 30° C. with initial OD₆₀₀ of ˜1.0 and under oxygen limited conditions.

Yeast Transformation

Transformation of expression cassettes for constructing xylose and cellobiose metabolic pathways was performed using the yeast EZ-Transformation kit (BIO 101, Vista, Calif.). All transformants were selected on YSC agar medium containing 20 g/L of glucose. Amino acids and nucleotides were added as necessary.

AEP Enzymatic Assay

Cell cultures were grown in culture tubes filled with 5 mL YP medium supplemented with 80 g/L cellobiose. The cells were grown at 30° C. at 250 rpm for 48 hours, and then resuspended in Y-PER Extraction Reagent (Thermal Scientific, Rockford, Ill.) following the manufacturer's instructions. Supernatants were then collected for measurement of protein concentration and AEP activity. To determine the total protein concentration, BCA Protein Assay Reagent (Thermal Scientific, Rockford, Ill.) was used following the manufacturer's instructions. A Synergy 2 Multi-Mode Microplate Reader was used to measure the absorbance change at OD₅₈₀. Total protein concentration was calculated following the manufacturer's instructions. To determine the AEP activity, the conversion between α-glucose and β-glucose is coupled to the oxidation of β-glucose catalyzed by β-D-glucose dehydrogenase and the reduction of NAD⁺. An assay mixture containing 0.34 mM NAD⁺, 0.5U of β-D-glucose dehydrogenase and 50 mM Tris/HCl buffer was prepared. 820 μL of the mixture was pipetted into a UV cuvette and then 130 μL AEP-containing solution was added. The reaction was initiated by the addition of 50 μL 166 μM freshly prepared α-glucose, and the increase in absorption at 340 nm was recorded for 3 minutes.

Analytical Methods

Cell growth was monitored by optical density (OD) at 600 nm using UV-visible Spectrophotometer (Biomate 5, Thermo, NY). Glucose, xylose, xylitol, glycerol, acetate and ethanol concentrations were determined by high performance liquid chromatography (HPLC, Agilent Technologies 1200 Series) equipped with a refractive index detector using a Rezex ROA-Organic Acid H+ (8%) column (Phenomenex Inc., Torrance, Calif.). The column was eluted with 0.005 N of H₂SO₄ at a flow rate of 0.6 ml/min at 50° C.

1. A host cell comprising:
a) recombinant DNA encoding one or more cellodextrin transporters;
b) recombinant DNA encoding one or more polypeptides having glucose mutarotase activity;
wherein the host cell consumes more molecules of cellobiose when grown in a cellobiose-containing medium than are consumed by a corresponding host cell lacking said recombinant DNA encoding one or more polypeptides having glucose mutarotase activity.

2. The host cell of claim 1, wherein the host cell further comprises recombinant DNA encoding one or more β-glucosidases.

3. The host cell of claim 1, wherein the polypeptide having glucose mutarotase activity is a polypeptide comprising an amino acid sequence selected from the group consisting of SEQ ID NOs: 17, 19, 21, and 23.

4. The host cell of claim 1, wherein the polypeptide having glucose mutarotase activity is a polypeptide comprising an amino acid sequence selected from the group consisting of SEQ ID NOs: 28-32.

5. A method of fermenting a cellobiose-containing mixture, the method comprising:
a) contacting the cellobiose-containing mixture with the host cell of claim 1, and
b) incubating the host cell under conditions that support fermentation.

6. (canceled)

7. A method of increasing production of a chemical, the method comprising:
a) providing a host cell, wherein the host cell comprises:
i) recombinant DNA encoding one or more cellodextrin transporters;
ii) recombinant DNA encoding one or more polypeptides having glucose mutarotase activity; and
b) culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased production of the chemical by the cell as compared with a corresponding host cell lacking said recombinant DNA encoding one or more polypeptides having glucose mutarotase activity.

8. The method of claim 7, wherein the chemical is an alcohol.

9. The method of claim 8, wherein the alcohol is selected from group consisting of: ethanol, n-propanol, n-butanol, iso-butanol, 3-methyl-1-butanol, 2-methyl-1-butanol, 3-methyl-1-pentanol, and octanol.

10. The method of claim 7, wherein the chemical is a terpenoid, a polyketide, a fatty acid, a fatty acid derivative, or an organic acid.

11. A method of increasing the growth rate of a cell, the method comprising:
a) providing a host cell, wherein the host cell comprises:
i) recombinant DNA encoding one or more cellodextrin transporters;
ii) recombinant DNA encoding one or more polypeptides having glucose mutarotase activity; and
b) culturing the host cell in a medium such that the recombinant DNA encoding the one or more cellodextrin transporters and the one or more polypeptides having glucose mutarotase activity are expressed, and wherein expression of the recombinant DNA encoding one or more polypeptides having glucose mutarotase activity results in increased growth rate of the cell as compared with a corresponding host cell lacking said recombinant DNA encoding one or more polypeptides having glucose mutarotase activity.

12. The method of claim 7, wherein the host cell further comprises recombinant DNA encoding one or more β-glucosidases.

13. The method of claim 7, wherein the polypeptide having mutarotase activity is a polypeptide comprising an amino acid sequence selected from the group consisting of SEQ ID NOs: 17, 19, 21, and 23.

14. The method of, claim 7, wherein the polypeptide having mutarotase activity is a polypeptide comprising an amino acid sequence selected from the group consisting of SEQ ID NOs: 28-32.

15. (canceled)

16. A method of fermenting a β-D-glucose-containing mixture, the method comprising:
a) providing a host cell, wherein the host cell comprises recombinant DNA encoding one or more polypeptides having glucose mutarotase activity; and
b) culturing the host cell in a medium such that the recombinant DNA encoding the one or more polypeptides having glucose mutarotase activity is expressed, and wherein expression of the recombinant DNA encoding one or more polypeptides having glucose mutarotase activity results in increased consumption of β-D-glucose-containing mixture by the cell as compared with a corresponding host cell lacking said recombinant DNA encoding one or more polypeptides having glucose mutarotase activity.

17. The method of claim 16, wherein the β-D-glucose-containing mixture is obtained from the hydrolysis of cellulose.

18. The method of claim 16, wherein the polypeptide having mutarotase activity is a polypeptide comprising an amino acid sequence selected from the group consisting of SEQ ID NOs: 17, 19, 21, and 23.

19. The method of claim 16, wherein the polypeptide having mutarotase activity is a polypeptide comprising an amino acid sequence selected from the group consisting of SEQ ID NOs: 28-32.

20. The host cell of claim 1 wherein the polypeptide having glucose mutarotase activity comprises one or both amino acid sequences of SEQ ID NO: 28 and 29.

21. The host cell of claim 20, wherein the polypeptide having glucose mutarotase activity comprises the amino sequences of SEQ ID NOs: 28 and 29.

22. The host cell of claim 20, wherein the host cell further comprises recombinant DNA encoding one or more β-glucosidases.


SEQUENCE LISTING (34 sequences)

SEQ ID NO: 1 - protein, 18 aa, Artificial Sequence
  Sequence motif
XYXXXXXXXXXXXXXXXD

SEQ ID NO: 2 - protein, 17 aa, Artificial Sequence
  Sequence motif
XXXGXXXXXXXXXXXXX

SEQ ID NO: 3 - protein, 3 aa, Artificial Sequence
  Sequence motif
GRX

SEQ ID NO: 4 - protein, 9 aa, Artificial Sequence
  Sequence motif
RXXXXXXXN

SEQ ID NO: 5 - protein, 8 aa, Artificial Sequence
  Sequence motif
WRXPXXXQ

SEQ ID NO: 6 - protein, 24 aa, Artificial Sequence
  Sequence motif
PESPRXLXXXXXXXXAXXXLXXYH

SEQ ID NO: 7 - protein, 9 aa, Artificial Sequence
  Sequence motif
FXQXSGNXX

SEQ ID NO: 8 - protein, 18 aa, Artificial Sequence
  Sequence motif
LXXXXXXEXLXXXXRXKG

SEQ ID NO: 9 - protein, 5 aa, Artificial Sequence
  Transmembrane helix
PESPR

SEQ ID NO: 10 - protein, 4 aa, Artificial Sequence
  Transmembrane helix
PETK

SEQ ID NO: 11 - protein, 18 aa, Artificial Sequence
  Sequence motif
XXXXXXXXGRXXXXXXXX

SEQ ID NO: 12 - protein, 26 aa, Artificial Sequence
  Sequence motif
XXGXXXGXXXXXXXXXXXXXXXXXXX

SEQ ID NO: 13 - protein, 8 aa, Artificial Sequence
  Synthesized Construct
XXVXXXXX

SEQ ID NO: 14 - protein, 9 aa, Artificial Sequence
  Sequence motif
XXXXENGXX

SEQ ID NO: 15 - protein, 18 aa, Artificial Sequence
  Sequence motif
XXXXXXXXXGXXXXDXXX

SEQ ID NO: 16 - DNA, 2100 bp, Saccharomyces cerevisiae
atgacagctcagttacaaagtgaaagtacttctaaaattgttttggttacaggtggtgctggatacattggttcacacactgtggtagagctaattgagaatggatatgactgtgttgttgctgataacctgtcgaattcaacttatgattctgtagccaggttagaggtcttgaccaagcatcacattcccttctatgaggttgatttgtgtgaccgaaaaggtctggaaaaggttttcaaagaatataaaattgattcggtaattcactttgctggtttaaaggctgtaggtgaatctacacaaatcccgctgagatactatcacaataacattttgggaactgtcgttttattagagttaatgcaacaatacaacgtttccaaatttgttttttcatcttctgctactgtctatggtgatgctacgagattcccaaatatgattcctatcccagaagaatgtcccttagggcctactaatccgtatggtcatacgaaatacgccattgagaatatcttgaatgatctttacaatagcgacaaaaaaagttggaagtttgctatcttgcgttattttaacccaattggcgcacatccctctggattaatcggagaagatccgctaggtataccaaacaatttgttgccatatatggctcaagtagctgttggtaggcgcgagaagctttacatcttcggagacgattatgattccagagatggtaccccgatcagggattatatccacgtagttgatctagcaaaaggtcatattgcagccctgcaatacctagaggcctacaatgaaaatgaaggtttgtgtcgtgagtggaacttgggttccggtaaaggttctacagtttttgaagtttatcatgcattctgcaaagcttctggtattgatcttccatacaaagttacgggcagaagagcaggtgatgttttgaacttgacggctaaaccagatagggccaaacgcgaactgaaatggcagaccgagttgcaggttgaagactcctgcaaggatttatggaaatggactactgagaatccttttggttaccagttaaggggtgtcgaggccagattttccgctgaagatatgcgttatgacgcaagatttgtgactattggtgccggcaccagatttcaagccacgtttgccaatttgggcgccagcattgttgacctgaaagtgaacggacaatcagttgttcttggctatgaaaatgaggaagggtatttgaatcctgatagtgcttatataggcgccacgatcggcaggtatgctaatcgtatttcgaagggtaagtttagtttatgcaacaaagactatcagttaaccgttaataacggcgttaatgcgaatcatagtagtatcggttctttccacagaaaaagatttttgggacccatcattcaaaatccttcaaaggatgtttttaccgccgagtacatgctgatagataatgagaaggacaccgaatttccaggtgatctattggtaaccatacagtatactgtgaacgttgcccaaaaaagtttggaaatggtatataaaggtaaattgactgctggtgaagcgacgccaataaatttaacaaatcatagttatttcaatctgaacaagccatatggagacactattgagggtacggagattatggtgcgttcaaaaaaatctgttgatgtcgacaaaaacatgattcctacgggtaatatcgtcgatagagaaattgctacctttaactctacaaagccaacggtcttaggccccaaaaatccccagtttgattgttgttttgtggtggatgaaaatgctaagccaagtcaaatcaatactctaaacaatgaattgacgcttattgtcaaggcttttcatcccgattccaatattacattagaagttttaagtacagagccaacttatcaattttataccggtgatttcttgtctgctggttacgaagcaagacaaggttttgcaattgagcctggtagatacattgatgctatcaatcaagagaactggaaagattgtgtaaccttgaaaaacggtgaaacttacgggtccaagattgtctacagattttcctga

SEQ ID NO: 17 - protein, 699 aa, Saccharomyces cerevisiae
MTAQLQSESTSKIVLVTGGAGYIGSHTVVELIENGYDCVVADNLSNSTYDSVARLEVLTKHHIPFYEVDLCDRKGLEKVFKEYKIDSVIHFAGLKAVGESTQIPLRYYHNNILGTVVLLELMQQYNVSKFVFSSSATVYGDATRFPNMIPIPEECPLGPTNPYGHTKYAIENILNDLYNSDKKSWKFAILRYFNPIGAHPSGLIGEDPLGIPNNLLPYMAQVAVGRREKLYIFGDDYDSRDGTPIRDYIHVVDLAKGHIAALQYLEAYNENEGLCREWNLGSGKGSTVFEVYHAFCKASGIDLPYKVTGRRAGDVLNLTAKPDRAKRELKWQTELQVEDSCKDLWKWTTENPFGYQLRGVEARFSAEDMRYDARFVTIGAGTRFQATFANLGASIVDLKVNGQSVVLGYENEEGYLNPDSAYIGATIGRYANRISKGKFSLCNKDYQLTVNNGVNANHSSIGSFHRKRFLGPIIQNPSKDVFTAEYMLIDNEKDTEFPGDLLVTIQYTVNVAQKSLEMVYKGKLTAGEATPINLTNHSYFNLNKPYGDTIEGTEIMVRSKKSVDVDKNMIPTGNIVDREIATFNSTKPTVLGPKNPQFDCCFVVDENAKPSQINTLNNELTLIVKAFHPDSNITLEVLSTEPTYQFYTGDFLSAGYEARQGFAIEPGRYIDAINQENWKDCVTLKNGETYGSKIVYRFS

SEQ ID NO: 18 - DNA, 1026 bp, Saccharomyces cerevisiae
atgtcaaataataaggctggcggtgaatatgaagtcatcactattggtgatgcaaaaaagcttcaagcaacaatttcagagcttggcgctactttgcttgacttgaaagtgaacaacgaatcgattgttctaggataccctgatattcatggctatatatcagatggctacaactacattggtgctactgttggccgttatgcaaatcgtatttacaagggcatgttcagtatggaagatggacctcatcagttgacagtgaacaattgtggaaacaccaaccacagtagcatcagctccttccatctcaagaagtataaggcatccaaagtccaaaatcctcttgatgacctttacattgttgaatttacgttattggatgaccgcacactgcccaatgaatttccgggagatctagctgtaaacttaaagtacacattgaacgttgctgacatgaccttggacttggaatacgaggcaaaactggtcagcggtgaggctactcccatcaacatgacgaatcacacgtacttcaatttaaacaaaactatggataaaaaatccattagcggtacggaggttaggctctgctcagataaatcattggaagtaagcgaaggagcactaattccaaccggtaaaatcgttcaaagaaaaattgctacttttgactcctccaaacctaccattctgcaggatgatggcccgatttatgactacgctttcattgttgatgaaaataagaatttgaaaacgacagactcagtaagtgtaaacaagctcgtccctgccttcaaggcttatcatccggcatcacgactttcattggaggtttcaacaacagaaccaactgtcctattttacacaggtgacaacttgtgtgatggatttacgccgagatctggctttgccgttgaacaaggcagatatgttgacgcaatcaaccgtgatggatggagagactgcgttcttttgagacgtggtgaggtatatacttctaagactcgatatcgttttgcggtgtag

SEQ ID NO: 19 - protein, 341 aa, Saccharomyces cerevisiae
MSNNKAGGEYEVITIGDAKKLQATISELGATLLDLKVNNESIVLGYPDIHGYISDGYNYIGATVGRYANRIYKGMFSMEDGPHQLTVNNCGNTNHSSISSFHLKKYKASKVQNPLDDLYIVEFTLLDDRTLPNEFPGDLAVNLKYTLNVADMTLDLEYEAKLVSGEATPINMTNHTYFNLNKTMDKKSISGTEVRLCSDKSLEVSEGALIPTGKIVQRKIATFDSSKPTILQDDGPIYDYAFIVDENKNLKTTDSVSVNKLVPAFKAYHPASRLSLEVSTTEPTVLFYTGDNLCDGFTPRSGFAVEQGRYVDAINRDGWRDCVLLRRGEVYTSKTRYRFAV

SEQ ID NO: 20 - DNA, 1029 bp, Saccharomyces cerevisiae
atgtcaaatagtaacggtgataataagtatggtgtcattacaattggtgatgagaaaaagttccaagctaccattgcgccacttggtgcaactttggtagacctgaaggtaaacggccaatcagtcgttcaaggttattcaaacgtacaagactatttaacagatggtaacatgatgggcgctacagttggtcgttatgccaaccgtatcgccaagggtgttttcagcctggatgatggtcctcacaagttaactgttaacaattgtggtaacacaaatcacagcagtatcagttccttaaatcttaagcagtacaaggcgtcccctgttgagaatccttccaagggcgtttacgttgttgaatttaaattgttggatgatcacacacagcccaaccctaacgagtttccaggtgacttggaagtcacagtgaagtacactttgaatgttgcagaaatgactttagacatggaataccaagcacagttagttcgtggtgatgctactccaattaatatgaccaaccactcgtattttaacttgaacaaagtcaagagtgagaagtccattcgtggtacagaagttaaggtctgctcgaacaaatcgcttgaagttacagagggggcattgcttccaactggtaagatcattgaaaggaacattgctacttttgattcaactaagcccaccgttttgcatgaagatactccggtctttgattgcactttcattattgacgctaacaaggatttgaagactacagattctgtgagtgtcaacaaattggttcctgtttttaaggcctatcatcccgaatctcacatcaaatttgaggtttcgacgacagaaccaaccgttcatttgtacaccggtgataatttatgtggcaaatttgtgccaaggtctggatttgccgttcagcaaggtagatacgttgatgctatcaaccgtgatgagtggagaggttgtgttcttctgaagcgcggcgaggtatacacttcaaagactcagtacaaattcgatatttag

SEQ ID NO: 21 - protein, 342 aa, Saccharomyces cerevisiae
MSNSNGDNKYGVITIGDEKKFQATIAPLGATLVDLKVNGQSVVQGYSNVQDYLTDGNMMGATVGRYANRIAKGVFSLDDGPHKLTVNNCGNTNHSSISSLNLKQYKASPVENPSKGVYVVEFKLLDDHTQPNPNEFPGDLEVTVKYTLNVAEMTLDMEYQAQLVRGDATPINMTNHSYFNLNKVKSEKSIRGTEVKVCSNKSLEVTEGALLPTGKIIERNIATFDSTKPTVLHEDTPVFDCTFIIDANKDLKTTDSVSVNKLVPVFKAYHPESHIKFEVSTTEPTVHLYTGDNLCGKFVPRSGFAVQQGRYVDAINRDEWRGCVLLKRGEVYTSKTQYKFDI

SEQ ID NO: 22 - DNA, 1053 bp, Neurospora crassa
atgtctgacgcaatcgcctccttcatccccttgggagccatcatccagtccctctacgtcgccggcaccaacatcgtcctcggcttcccctcccaaaaggcttacgagtcgcacaatgcctgcttcttcggcgagaccatcggccgcgtcgccaaccgggtcaagggcgccaagatcgactccctcaacggcgggcaatcgtacaccctggccgccaacaacgggcccaacaatctgcacggcggcgtggtcggctggggcaagaaggtctgggacggtcccaagcccgtcggcgtcagggagatccccgcgttcgagggcaagagcgacaagattgacggtggtgagagcgtccagttcacgctggtgagcgaggacggcgacgagggttttcccggaaaagtagaggctagggtgatctacaccgttagcaaggagaagggcgaggatggcaaggagaaggttttgcttggtattgagtatgaggcggagctgttggatggtgaggtggaggagacggtcatcaacatgactaaccattcatatttcaacctgaccggcggcccttccatcgaaggcaccataatcaccctcgccacgaaccagtacctccccgtcgaccaaggcggcatccccaccggcggccccgtgccctaccccaaccctgccgtcaagaatggcaacatcccattcaccctcggcgcggctgagcccgacatcgatgactgcttcatcgtcaacgaggacccttcgtcggttcccatcgatacgcgctcgctgcctctcaagaagaacctcagcgcgttccaccccgagtccaagatccatcttgaggtgtacagcaccgagcccgccttccagttttacacgggcaagtatattgacattcccgcggtagacggcgttcccgctcgcaagccgcgcagcggcttctgccttgagcccagcaggtgggtcaacgcttgcaatgtgcccgagtggaaggggcagatgttgttgaagaagggagagaagtatggatgcaagatcctgtacagggcgtggaaggagtag

SEQ ID NO: 23 - protein, 350 aa, Neurospora crassa
MSDAIASFIPLGAIIQSLYVAGTNIVLGFPSQKAYESHNACFFGETIGRVANRVKGAKIDSLNGGQSYTLAANNGPNNLHGGVVGWGKKVWDGPKPVGVREIPAFEGKSDKIDGGESVQFTLVSEDGDEGFPGKVEARVIYTVSKEKGEDGKEKVLLGIEYEAELLDGEVEETVINMTNHSYFNLTGGPSIEGTIITLATNQYLPVDQGGIPTGGPVPYPNPAVKNGNIPFTLGAAEPDIDDCFIVNEDPSSVPIDTRSLPLKKNLSAFHPESKIHLEVYSTEPAFQFYTGKYIDIPAVDGVPARKPRSGFCLEPSRWVNACNVPEWKGQMLLKKGEKYGCKILYRAWKE

SEQ ID NO: 24 - DNA, 2624 bp, Pichia stipitis
agcagtcacctagtaatcttggacagcttttggcaccccaacagcgacaatactagcgttttaactgtttgaggtctcttcttttgagataacacgcttaaacagcaaacagacaaaaatgtttaacggccaaattgcctgacaataggcacaccccacagagtcgtatgcacgggtataccccaggcggttgaaaaaaagtgtaacgttacagtgttcgcggccatagtcagaggttcttgctcttcgaccttcttgcagtccgagttggagaatcttcaatatattccaacacatttatcccaaaacacacacgaaacgaaaagttttcaccttgttattctttctattcatcgtcagctttcagaacaagttcttccttgaaaagctttttctatcagattagcgtctattgaaacctgattcagaactgaaaaatttcactcccatgtctgctccgcaagattacatcctcatcactggtggtgctggttacattggatcgcacaccgtgatcgaattggtaaacaacggctacaaggttgttgtagtcgactctcttgttaactcttcctatgatgctattgcaagaatcgagtacatcgttaagcagcctgtaccattctaccaggtcgacattagagacggcgaaaaattgaacgaagtgttccaaaagcacaaaattaccggtgttatccactttgctgccttgaaggctgttggagaatctactgagattcctttggaatactacgacaacaacgttaacggtactatcagcttgttgcaggtaatgaacaagaataacgtcaagtcgatcgttttctccagttcagccactgtctacggagacgttaccaggttcggagacaattctatgattccaattccagaacattgtccaactgaccctactaacccttacggaagaaccaagttagtcattgagaacatcttgagagatgtccactcttcttcttctctggagtggaaggttgctgtgttgagatacttcaaccctattggcgctcatccttctggtttaatcggcgaagatcctttaggtatccccaacaatttattgccttacctagcacaggttgctattggcagacgtgagaagttgtctgtattcggttctgactacgcttctcatgatggaactcctatcagggactatatccatgtcgtagacttggccaagggtcacctcgctgccttgagtttcttgaacaagactaatgaaggatctttccgcgaatggaacttaggcactggtaagggttctactgtcttcgacgtctaccacgcattcagcaaggctgtaggcagagacttaccttacgaggttgtgggtagaagagctggtgatgtgctcgatttgactgccaatcctactagagccaacaaggaattggaatggaaggccgagttgaccattgaagacgcctgtaaggacttgtggaaatggaccaccggaaacccaactggcttcaagatcgaaggctacgaatggaagttgttcgaaactgaagggcatgccaacagattacacaccttcactactggagacgttaccattgcattggctaacagaggtgctcttatccaagacctcagcttccaaggtgaacacttggtttgtgcctacgacaatggcgacgactacaagcttccaaccaacccattctttggtacaaatgttggccgtgtagccaacagaatcgccaacggtgagtttaagttgaacggtcagacctacaagacgccaaagaacgagaacaacaacaccttacacggaggtcttgttggtttcgacaagaggaactggttgggtcctgttatcagcaagaaggaagatgtgttcacagtcaagtttgctctcattgacgaagacggtcacaatggcttcccaggtgacttacagaccatcatcacctacacgattgaacctactgccgtcagtcatgagtacgaagccaagttgatcaacgataaagtggatgccacaatcgtcaacttgaccaaccactcgtacttcaacctctccggtgaacctacgaccgatgacactgatgtcaccttgttttacgacaagtacatggaagtcgatgataagacgttgttgccaactggtaacctcatcaagtatccacatgctactgccaacgtgccagttcacttggaccaagacgctttcttcgactactgtttcattgtcgacgaagatggctgtggtatcgataccagaaagtcgcagttaaagcctattttcaaggctacccacccaagagccaacaccgagttcctcttctcttctactgagccatctttccagttctatactggtgacggtgttaacaccaagggcttcgtcaagagaagtggtttctgtatcgagccagccagattcgttgattccatctcttttgagaaatgggctccacaagtcatcttgaagaagggtgaaacttatggttctctcacgaagtactccttctctaagaagcaatgagtcagataaaaaactttacactaggtaggaaaaaaaaatagattttgatgaagattgactttcataaccggtcaatattacgctgagtaggaataaaatagactttcat

SEQ ID NO: 25 - protein, 688 aa, Pichia stipitis
MSAPQDYILITGGAGYIGSHTVIELVNNGYKVVVVDSLVNSSYDAIARIEYIVKQPVPFYQVDIRDGEKLNEVFQKHKITGVIHFAALKAVGESTEIPLEYYDNNVNGTISLLQVMNKNNVKSIVFSSSATVYGDVTRFGDNSMIPIPEHCPTDPTNPYGRTKLVIENILRDVHSSSSSEWKVAVLRYFNPIGAHPSGLIGEDPLGIPNNLLPYLAQVAIGRREKLSVFGSDYASHDGTPIRDYIHVVDLAKGHLAALSFLNKTNEGSFREWNLGTGKGSTVFDVYHAFSKAVGRDLPYEVVGRRAGDVLDLTANPTRANKELEWKAELTIEDACKDLWKWTTGNPTGFKIEGYEWKLFETEGHANRLHTFTTGDVTIALANRGALIQDLSFQGEHLVCAYDNGDDYKLPTNPFFGTNVGRVANRIANGEFKLNGQTYKTPKNENNNTLHGGLVGFDKRNWLGPVISKKEDVFTVKFALIDEDGHNGFPGDLQTIITYTIEPTAVSHEYEAKLINDKVDATIVNLTNHSYFNLSGEPTTDDTDVTLFYDKYMEVDDKTLLPTGNLIKYPHATANVPVHLDQDAFFDYCFIVDEDGCGIDTRKSQLKPIFKATHPRANTEFLFSSTEPSFQFYTGDGVNTKGFVKRSGFCIEPARFVDSISFEKWAPQVILKKGETYGSLTKYSFSKKQ

SEQ ID NO: 26 - DNA, 1041 bp, Escherichia coli
gtgctgaacgaaactcccgcactggcacccgatggtcagccgtaccgactgttaactttgcgtaacaacgcagggatggtagtcacgctgatggactggggtgcgactttactttccgcccgtattccgctttccgatggcagcgtccgcgaggcgctgctcggctgtgccagcccggaatgctatcaggatcaggccgcgtttctgggggcctctattggtcgttatgccaaccgtatcgccaatagccgttatacctttgacggtgaaaccgtgacgctttcgccaagtcagggcgttaaccagctgcacggcgggccggaagggttcgacaaacgtcgctggcagattgtgaaccagaacgatcgtcaggtgctgtttgccctgagttcagatgatggtgatcagggcttcccgggtaatctcggcgcgacggtgcaatatcgtctgaccgacgataaccgtatctccattacttatcgcgccacagttgataaaccttgcccggtgaatatgactaatcacgtctatttcaatcttgacggcgagcagtctgacgtgcgcaatcacaagttgcagattctggcggacgaatatctgccggttgatgaaggcggcattccgcacgacggcctgaaatctgtcgccggaacgtcttttgatttccgcagcgccaaaatcatcgccagtgagtttcttgccgacgacgatcagcgcaaagtgaaaggttacgatcacgcattcttgttacaggccaaaggcgatggcaagaaagtggcggcgcatgtctggtcagcagatgaaaaattgcagctgaaggtctacaccaccgctccggctctgcaattctactccggcaacttcctcggcggcacaccgtcgcggggaaccgaaccttacgccgactggcaagggctggctctggaaagcgagtttctaccggacagcccgaaccaccctgaatggccgcaaccggactgcttcctgcgtcctggcgaagagtattccagcctgacggaatatcagtttattgctgagtaa

SEQ ID NO: 27 - protein, 346 aa, Escherichia coli
MLNETPALAPDGQPYRLLTLRNNAGMVVTLMDWGATLLSARIPLSDGSVREALLGCASPECYQDQAAFLGASIGRYANRIANSRYTFDGETVTLSPSQGVNQLHGGPEGFDKRRWQIVNQNDRQVLFALSSDDGDQGFPGNLGATVQYRLTDDNRISITYRATVDKPCPVNMTNHVYFNLDGEQSDVRNHKLQILADEYLPVDEGGIPHDGLKSVAGTSFDFRSAKIIASEFLADDDQRKVKGYDHAFLLQAKGDGKKVAAHVWSADEKLQLKVYTTAPALQFYSGNFLGGTPSRGTEPYADWQGLALESEFLPDSPNHPEWPQPDCFLRPGEEYSSLTEYQFIAE

SEQ ID NO: 28 - protein, 11 aa, Artificial Sequence
  Sequence motif
GXXXGRXXNRX

SEQ ID NO: 29 - protein, 13 aa, Artificial Sequence
  Sequence motif
TXXXXXXHXYXNL

SEQ ID NO: 30 - protein, 322 aa, Saccharomyces cerevisiae
IGAGTRFQATFANLGASIVDLKVNGQSVVLGYENEEGYLNPDSAYIGATIGRYANRISKGKFSLCNKDYQLTVNNGVNANHSSIGSFHRKRFLGPIIQNPSKDVFTAEYMLIDNEKDTEFPGDLLVTIQYTVNVAQKSLEMVYKGKLTAGEATPINLTNHSYFNLNKPYGDTIEGTEIMVRSKKSVDVDKNMIPTGNIVDREIATFNSTKPTVLGPKNPQFDCCFVVDENAKPSQINTLNNELTLIVKAFHPDSNITLEVLSTEPTYQFYTGDFLSAGYEARQGFAIEPGRYIDAINQENWKDCVTLKNGETYGSKIVYRFS

SEQ ID NO: 31 - protein, 339 aa, Saccharomyces cerevisiae
EARFSAEDMRYDARFVTIGAGTRFQATFANLGASIVDLKVNGQSVVLGYENEEGYLNPDSAYIGATIGRYANRISKGKFSLCNKDYQLTVNNGVNANHSSIGSFHRKRFLGPIIQNPSKDVFTAEYMLIDNEKDTEFPGDLLVTIQYTVNVAQKSLEMVYKGKLTAGEATPINLTNHSYFNLNKPYGDTIEGTEIMVRSKKSVDVDKNMIPTGNIVDREIATFNSTKPTVLGPKNPQFDCCFVVDENAKPSQINTLNNELTLIVKAFHPDSNITLEVLSTEPTYQFYTGDFLSAGYEARQGFAIEPGRYIDAINQENWKDCVTLKNGETYGSKIVYRFS

SEQ ID NO: 32 - protein, 336 aa, Saccharomyces cerevisiae
FSAEDMRYDARFVTIGAGTRFQATFANLGASIVDLKVNGQSVVLGYENEEGYLNPDSAYIGATIGRYANRISKGKFSLCNKDYQLTVNNGVNANHSSIGSFHRKRFLGPIIQNPSKDVFTAEYMLIDNEKDTEFPGDLLVTIQYTVNVAQKSLEMVYKGKLTAGEATPINLTNHSYFNLNKPYGDTIEGTEIMVRSKKSVDVDKNMIPTGNIVDREIATFNSTKPTVLGPKNPQFDCCFVVDENAKPSQINTLNNELTLIVKAFHPDSNITLEVLSTEPTYQFYTGDFLSAGYEARQGFAIEPGRYIDAINQENWKDCVTLKNGETYGSKIVYRFS

SEQ ID NO: 33 - protein, 579 aa, Neurospora crassa
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 34 - protein, 525 aa, Neurospora crassa
MGIFNKKPVAQAVDLNQIQEEAPQFERVDWKKDPGLRKLYFYAFILCIASATTGYDGMFFNSVQNFETWIKYFGDPRGSELGLLGALYQIGSIGSIPFVPLLTDNFGRKTPIIIGCVIMIVGAVLQATAKNLDTFMGGRTMLGFGNSLAQIASPMLLTELAHPQHRARLTTIYNCLWNVGALVVSWLAFGTNYINNDWSWRIPALLQAFPSIIQLLGIWWVPESPRFLIAKDKHDEALHILAKYHANGDPNHPTVQFEFREIKETIRLEMESTKNSSYLDFFKSRGNRYRLAILLSLGFFSQWSGNAIISNYSSKLYETAGVTDSTAKLGLSAGQTGLALIVSVTMALLVDKLGRRLAFLASTGGMCGTFVIWTLTAGLYGEHRLKGADKAMIFFIWVFGIFYSLAWSGLLVGYAIEILPYRLRGKGLMVMNMSVQCALTLNTYANPVAFDYFGPDHSWKLYLIYTCWIAAEFVFVFFMYVETKGPTLEELAKVIDGDEADVAHIDIHQVEKEVEIHEHEGKSVA